(19) United States
(12) Patent Application Publication
(10) Pub. No.: US 2024/0401022 A1
(43) Pub. Date: Dec. 5, 2024

(54) DEVICES, METHODS, AND SYSTEMS FOR EXTRACTING GENOMIC DNA FROM CELL SAMPLES WITH TUNABLE SIZE SELECTION

(71) Applicant: INSO BIOSCIENCES INC., Ithaca, NY (US)

(72) Inventors: Adam Bisogni, Ithaca, NY (US); Harold Craighead, Ithaca, NY (US); Harvey Tian, Ithaca, NY (US)

(21) Appl. No.: 18/690,011

(22) PCT Filed: Sep. 9, 2022
(86) PCT No.: PCT/US2022/043093

§ 371 Date: Mar. 7, 2024

Related U.S. Application Data
(60) Provisional application No. 63/242,216, filed on Sep. 9, 2021.
(60) Provisional application No. 63/320,389, filed on Mar. 16, 2022.

(51) Int. Cl. C12N 15/10; B01L 3/00

(52) U.S. Cl. CPC C12N 15/1003; B01L 3/502753; B01L 2200/025; B01L 2200/0647; B01L 2200/12

(57) ABSTRACT

Disclosed are microfluidic flow-based devices for extracting fragments of genomic DNA in a selected size range from a cell or cell nucleus. The devices include a microfluidic channel and an array of micropillars disposed within the microfluidic channel in a defined configuration. Also disclosed are systems including such devices and a fluid control module. Additionally disclosed are methods of selecting parameters for extracting fragments of genomic DNA having a desired size metric from a cell or cell nucleus, as well as methods of isolating fragments of genomic DNA having a selected size metric from a cell or cell nucleus.

### CROSS-REFERENCE TO RELATED APPLICATIONS

This application claims the benefit of U.S. Provisional Application No. 63/242,216, filed on Sep. 9, 2021; and U.S. Provisional Application No. 63/320,389, filed on Mar. 16, 2022, the entire contents of which are incorporated herein in their entirety by this reference.

### STATEMENT OF RIGHT

This invention was made with government support under grant number 1940395 awarded by The National Science Foundation. The government has certain rights in the invention.

### BACKGROUND

Extracting DNA, such as genomic DNA, from samples containing cells is necessary for a variety of applications. For example, extracted DNA would be needed for pulse-field gel electrophoresis, amplification, and detection of specific loci, or sequencing, among many other applications. These applications, in turn, can be useful for understanding important phenomena, such as cancer, infectious diseases, and many others.

Using microfluidic devices is one of the options for extracting DNA from cells. For instance, one may use micropillar arrays within a device to immobilize genomic DNA (gDNA) from lysed cells, and then to extract that gDNA from the arrays. Despite having numerous advantages, these methods often yield a mixture of varying gDNA fragment sizes and often allow processing a limited range of samples.

Therefore, there is a need in the art to further develop methods for extracting gDNA from cellular samples.

### SUMMARY

This invention relates, in some aspects, to methods for extracting and isolating genomic DNA (gDNA) with selectable fragment sizes from samples comprising cells or cell nuclei. The extracted DNA may be analyzed by a variety of techniques such as fragment sizing, selective amplification, or sequencing. This capability is of value for biological research and for medical uses such as disease diagnosis and direction of therapies. Selecting the optimum gDNA fragment size for a particular sequencing technique improves the efficiency of the process. The disclosed approach should also reduce the number of steps required to prepare samples and be efficient for small sample sizes.

To extract gDNA from selected cells or cell nuclei at select fragment sizes, enzymatic approaches are often utilized to shear the DNA into desired sizes, releasing the shorter DNA fragments from pillar-like obstructions in a microfluidic device in which they were immobilized. The gDNA can then be removed from the device or undergo further processing such as exchanging buffer or concentration in a second stage of the device. The gDNA is then compatible for analytical processes such as selective amplification, detection, or sequencing.

Methods for fabricating the microfluidic device employ standard approaches of lithography and etching to create molds that can be replicated in Polydimethylsiloxane, (PDMS) or other polymers using molding, embossing or injection molding. An example for a method to fabricate the device is described in Benitez et al. The molded PDMS can be easily bonded to a glass plate to complete the structure, or the device may be constructed by a combination of different polymeric materials.

Alternatively, the microfluidic device can be fabricated with other materials. For example, in addition to a polymer (e.g., PDMS), materials such as glass, plastics, metals, silicons, or any combination thereof can be used.

The combination of channel dimensions, pillar sizes, and the organization of the pillar array can be varied for a desired type of sample or sample volume. For example, smaller pillar spacings would be used to trap smaller cells or cell nuclei while larger spacings would be used to select larger cells from a mixture containing smaller entities in the sample.

In some aspects, microfluidic flow-based devices for extracting fragments of genomic DNA in a selected size range from a cell or cell nucleus comprise a microfluidic channel having an inlet port and an outlet port to allow a flow in a flow direction from the inlet port toward the outlet port. In preferred embodiments, the device comprises an array of micropillars disposed within the microfluidic channel.

In some embodiments, the micropillars have diameters preferably between at least about 3 micrometers and about 15 micrometers, but could range from at least about 1 micrometer to about 100 micrometers. In some embodiments, the diameter is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 micrometers; or any range between these values.

In some embodiments, the micropillars have a preferable height of at least about 15-about 25 micrometers but could range from at least about 1 to about 200 micrometers. In some embodiments, the height is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 micrometers; or any range between these values.

In some embodiments, the micropillars are separated from each other by spacings preferably between at least about 2 micrometers and about 150 micrometers, but could range from 0.1 micrometers up to more than 300 micrometers. In some embodiments, the spacing is at least about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 micrometers; or any range between these values.

In some embodiments, each array of micropillars is bounded within an area of 25 square microns or greater. In some embodiments, each array of micropillars is bounded within an area of 1 square millimeter or greater. In some embodiments, the area is at least about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 square millimeters; or any range between these values. In some embodiments, each array of micropillars is bounded within an area of less than about 200 square millimeters. In some embodiments, each array of micropillars is bounded within an area of less than about 100 square millimeters. In preferred embodiments, each array of micropillars is bounded within an area of less than about 1600 square centimeters.

In some embodiments, the micropillars are arranged within the array in a defined configuration (e.g., as shown in FIG. 9 and FIG. 10).

In some embodiments, the micropillars comprise a polymer (e.g., they are made of a polymer), plastics, metals, silicones, glass, or any combination of two or more thereof. In some embodiments, the micropillars are fabricated using a lithographic process. In some embodiments, the device further comprises one or more canals extending to within said area of the array of micropillars. In some embodiments, the one or more canals are aligned along the flow direction. In some embodiments, said diameters, said spacings, said area, said configuration, or a combination thereof is selected based on type of said cell or cell nucleus. In some embodiments, said cell is a leukocyte. In some embodiments, said cell is a mammalian cell. In some embodiments, said cell is a T cell. In some embodiments, said nucleus is a plant cell nucleus (or a cell nucleus from another organism). In some embodiments, said configuration allows extracting fragments of genomic DNA from a single cell by virtue of said configuration being narrower near the inlet port. In some embodiments, said configuration allows extracting fragments of genomic DNA from a plurality of cells or cell nuclei. In some embodiments, the device further comprises an agent that binds to the cell or nucleus. In some embodiments, the agent is an antibody, an antigen-binding fragment thereof, or an aptamer. In some embodiments, the device further comprises a secondary channel perpendicular to the microfluidic channel. Alternatively, the secondary channel that crosses the first channel can be at any angle. In some embodiments, the secondary channel allows lysing the cell after the cell is bound to the agent. In some embodiments, the micropillars comprise polydimethylsiloxane.

In some aspects, systems comprising any embodiment of these devices further comprise a fluid control module adapted to connect to the inlet port.

In some embodiments, the fluid control module comprises a pipette or a syringe. In some embodiments, the fluid control module comprises pressurized air source (e.g., compressed air), controlled pressure source, controlled pneumatic pressure source, pressure driven pump, syringe pump, vacuum pump, or a peristaltic pump. In some embodiments, the system further comprises one or more collection reservoirs adapted to connect to the outlet port. In some embodiments, the one or more collection reservoirs comprise a sample collection reservoir and a waste collection reservoir. In some embodiments, the system further comprises a controlled voltage source for electrophoretically driving DNA within the microfluidic channel. In some embodiments, the system comprises a plurality of the arrays of micropillars within a plurality of the microfluidic channels. In some embodiments, the arrays are in a staggered configuration in the device. In some embodiments, the arrays are in an ordered configuration that aligns with a multi-well plate configuration. In some embodiments, said multi-well plate is a 6-well, 12-well, 24-well, 48-well, 96-well, 384-well plate, 1536-well, or other similar formats. In some embodiments, the fluid control module allows delivering the cell, a lysis buffer, a digestion buffer comprising one or more enzymes, and a wash buffer into the microfluidic channel. In some embodiments, the system further comprises said digestion buffer, wherein the one or more enzymes comprise one or more restriction enzymes. In some embodiments, the system further comprises said digestion buffer, wherein the one or more enzymes comprise one or more nucleases, such as endonucleases.

In some aspects, methods of selecting parameters for extracting fragments of genomic DNA having a desired size metric from a cell or cell nucleus comprise processing a sample containing at least one cell or cell nucleus through the system of the present disclosure, at least once using parameters that comprise a concentration for each of said one or more enzymes; a number of said one or more enzymes; a buffer composition for said digestion buffer; a digestion time; and a digestion temperature; determining a fragment size metric for a collected sample; and, based on said fragment size metric, if said fragment size metric corresponds to the desired size metric, selecting the used parameters; if said fragment size metric is lesser than the desired size metric, reducing said concentration, reducing said number, reducing said digestion time, changing said digestion temperature to reduce enzyme efficiency, changing said buffer composition to reduce enzyme efficiency, or a combination thereof until said fragment size metric is not lesser than the desired size metric; and if said fragment size metric is greater than the desired size metric, increasing said concentration, increasing said number, increasing said digestion time, changing said digestion temperature to increase enzyme efficiency, changing said buffer composition to increase enzyme efficiency, or a combination thereof until said fragment size metric is not greater than the desired size metric.

In some embodiments, said fragment size metric is fragment size mean or fragment size median. In some embodiments, said processing comprises loading the sample into the device and washing at least one cell or cell nucleus in the device using the wash buffer. In some embodiments, the methods further comprise inactivating the one or more enzymes. In some embodiments, said inactivation comprises heat inactivation.

In some aspects, methods of isolating fragments of genomic DNA having a selected size metric from a cell or cell nucleus comprise processing a sample containing at least one cell or cell nucleus through the system of the present disclosure, using parameters selected via the methods described herein; and collecting the fragments of genomic DNA isolated via said processing.

In some embodiments, the methods further comprise recovering the collected fragments. In some embodiments, said recovering comprises removing the fragments from the collection reservoir via manual pipetting or through tubing. In some embodiments, said recovering comprises removing the fragments from the collection reservoir via an electrophoretic channel connected to the collection reservoir.

### BRIEF DESCRIPTION OF FIGURES

FIG. 1. Workflow overview of utilizing an exemplary embodiment of the disclosed system for sample prep for genomic analysis. Here, 4 key steps outline the overall workflow of utilizing the disclosed platform technology for cellular sample prep. These can be generally described as (1) obtaining a biological sample comprising at least one cell, e.g., whole blood or cell samples in a solution, (2) loading the sample onto a device of the present disclosure, which will then be loaded into a platform instrument responsible for fluid control and user interfacing, (3) removing the post-processed/prepped samples from the platform and then analyzing the sample, and (4) arriving at the data analysis and conclusion derived from step 3.

FIG. 2A-D. An exemplary workflow overview of micropillar array technology used for the described method of tuning gDNA size fragments collected from cellular gDNA extractions. Here, the 4-step genomic DNA (gDNA) extraction process performed by the micropillar array technology starts by (A) immobilizing cells within a sample, (B) chemically lysing the cell with lysis buffers, such as with a surfactant or high-salt buffer, (C) allowing the gDNA to become physically entangled within the immediate pillars and then either processing the gDNA while entangled on the micropillar array and/or fluorescently staining the gDNA for imaging, and finally (D) cleaving the gDNA into smaller fragments for release from the pillar array.

FIG. 3. Micrograph of genomic DNA extracted using the micropillar array technology. The genomic DNA has been stained with a fluorescent intercalating dye and is being visualized through fluorescence microscopy. FIG. 3 shows a device comprising a hexagonal lattice (staggered orientation), with a changing ‘Stepped’ gradient of pillar-to-pillar spacing with the number of pillars increases in density (decrease in micropillar spacing) in the direction towards an outlet port.

FIG. 4. Workflow illustration of target cell capture and subsequent gDNA extraction. Non-targeted cells (red) are able to flow through the larger structure array unhindered, and are not lysed with the target cells (purple). Targeted cells that become arrested on the surface of the larger pillars (at the intersection) due to their surface-binding characteristics, can then be lysed in the perpendicular direction to undergo gDNA extraction as described in FIG. 2 and FIG. 3. While the secondary channel that is in the perpendicular direction is shown as an example, the secondary channel that crosses the first channel can be at any angle with respect to the first channel.

FIG. 5A-C. An exemplary single cell workflow illustration. The general steps here are identical to those in FIG. 2, but the main difference is in channel structure. A solid wall structure enclosing the micropillar array while leaving only a single-cell sized opening at the apex of the array allows only a single cell to be loaded into each micropillar array region while additional cells are funneled via side “gutter” channels to load into further downstream pillar arrays or discarded, depending on application needs. The two panels on the right are micrographs showing a single human cancer cell loaded into the apex of the micropillar array, and the gDNA of single cell imaged post-lysis and staining.

FIG. 6. Varying pillar diameters for the micropillars within the array portion of the microfluidic chip. (Dimensions are shown in microns). Here, 4 separate examples of pillar diameter size are shown, ranging from ˜4 μm to ˜6.4 μm.

FIG. 7. Varying pillar-to-pillar spacings within the pillar array portion of the microfluidic chip. (Dimensions are shown in microns). Here, pillar-to-pillar spacing of 2.8 μm to 105 μm are shown. These can be freely adjusted as desired, depending on the cell size of a particular application.

FIG. 8 Varying micropillar region sizes within the microfluidic chip. (Measurements are shown in microns). Varying the length and width of the micropillar array regions can be done to accommodate varying cellular input sizes, ranging from single cells applications up to applications that need hundreds of thousands of cells per sample.

FIG. 9. Varying chip configurations, microchannel distribution & overall layout of the microfluidic channels. Orientation and size of each channel is freely adjustable, and individual channels can be fabricated separately than the other channels on the same silicon wafer master.

FIG. 10. 96-well ‘microplate’ configuration & layout of the microfluidic channels. Spacing channels so that the input ports and output ports line up with the spacing of individual wells in a 96-well microplate allows for a configuration that allows a total of 48 channels per microplate. This allows operation of the disclosed technology in standardized microplate/96-well format while retaining the functionality of the disclosed micropillar technology. Each of the 48 channels would contain a single or set of micropillar array channels and can be then operated in a fluidic system that can either control all 48 channels simultaneously or address each channel individually. The freedom and flexibility of operation can be dependent on application.

FIG. 11. Photograph of a typical microfluidic chip device with inlet tubing attached.

FIG. 12. Schematic diagram of the pressure driven flow system. A pressurized air source connects to the device through tubing, which pushes solutions through the chip and to the output port for collection.

FIG. 13. Schematic diagram of a fluid control system for introducing and recovering fluid from a microfluidic chip.

FIG. 14. Schematic diagram of a fluid control with an alternate arrangement of fluid control, indicating different types of pump types and valve configurations can be used.

FIG. 15. Schematic diagram of a fluid control system using applied electrical potential to drive charged molecules such as DNA or RNA, combined with conventional pumps for driving fluids.

FIG. 16. Schematic diagram of a fluid control system in which different output fluid components can be directed to different collection volumes.

FIG. 17. Flow chart showing the method for obtaining DNA fragments of a selected size. Using the disclosed methods, adjustments to DNA fragment size can be tuned to by altering conditions on the disclosed devices.

FIG. 18. Pulsed-field capillary electrophoresis analysis using the Femto Pulse system reveals of the size distribution of fragmented gDNA generated from mammalian cells using the disclosed technology. The particular conditions used in this sample produces a tight size range with a major peak at 8 kb.

FIG. 19. Pulsed-field capillary electrophoresis analysis using the Femto Pulse system reveals of the size distribution of fragmented gDNA generated from mammalian cells using the disclosed technology. The particular conditions used in this sample produces a tight size range with a major peak at 10 kb.

FIG. 20. Pulsed-field capillary electrophoresis analysis using the Femto Pulse system reveals of the size distribution of fragmented gDNA generated from mammalian cells using the disclosed technology. The particular conditions used in this sample produces a tight size range with a major peak at 14 kb.

FIG. 21. Pulsed-field capillary electrophoresis analysis using the Femto Pulse system reveals of the size distribution of fragmented gDNA generated from mammalian cells using the disclosed technology. The particular conditions used in this sample produces tight size range with a major peak at 25 kb.

FIG. 22. Pulsed-field capillary electrophoresis analysis using the Femto Pulse system reveals of the size distribution of fragmented gDNA generated from mammalian cells using the disclosed technology. The particular conditions used in this sample produces a broader size range, with a major peak around 200 kb.

FIG. 23A-B. Built in “canal” structures to facilitate cell loading further/deeper into the micropillar array region. (A) Shows a normal micropillar array region, where micropillars are spaced uniformly in columns that become denser and denser in spacing as one progresses further down the channel in the direction of flow. In this scenario, cells loaded may potentially localize within similar regions in the pillar array and therefore cause flow obstructions or clogging. In (B), canals are built into the pillar array region to allow cells to directly pass through earlier portions of the pillar array in order to spread out the final cell loading. This facilitates both cell lysis as well as eventual DNA processing by preventing the extracted gDNA from bundling into thicker “ropes”.

FIG. 24. Staggered micropillar array regions in a microfluidic device. This image depicts one possible such configuration of micropillar arrays within a device, in which multiple pillar arrays are staggered so as to improve the cell loading but spreading cells out over larger areas rather than having cells all become arrested and localized within a small area.

FIG. 25A-B. An exemplary microfluidic device. (A) 2 parallel microfluidic channels shown as a cropped-image, out of 8 total channels for this device design, where flow direction runs from up-to-down in orientation. (B) shows that the micropillars are arranged in what is designated herein as a “square” lattice, whereby pillars are placed in a way to have clear rows and columns at specified distances apart from each other. A “smooth” gradient of varying spacings of the pillars in square-lattice formation then defines the micropillar array regions. The micropillar channels are identical to one another, and the purpose of the 8 parallel channels it also meant to serve as an alternative to making one large giant channel.

FIG. 26A-C. An exemplary microfluidic device. (A) Similar to FIG. 25, 2 parallel microfluidic channels are shown as a cropped-image, out of 8 total channels for this device design, where flow direction runs from up-to-down in orientation. (B) and (C) show that the square-lattice micropillar array has differing spacings, as purposefully designed and can be changeable to any other desired spacing or dimensions. Unlike in FIG. 25, this device a “stepped” gradient in the channel rather than “smooth” gradient, where spacings are held uniform over a periodicity before they are changed to the next periodicity, rather than having one smooth transition of spacings from the top of the micropillar array to the bottom. Again, the micropillar channels are identical to one another, and the purpose of the 8 parallel channels it also meant to serve as an alternative to making one large giant channel.

FIG. 27A-C. An exemplary microfluidic device. (A) Similar to FIG. 25 and FIG. 26, 2 parallel microfluidic channels are shown as a cropped-image, out of 8 total channels for this device design, where flow direction runs from up-to-down in orientation. (B) and (C) show that the “hexagonal” lattice micropillar array has differing spacings, as purposefully designed and can be changeable to any other desired spacing or dimensions. Similar to FIG. 25 and FIG. 26, the gradient of pillar spacings can be either smooth or stepped. Also similar, the 2 micropillar channels are identical to one another compared with the other 6 of 8 total parallel channels, and again serves as an alternative to making one large giant channel.

FIG. 28A-F. An exemplary microfluidic device. (A) Flow direction goes from up-to-down, and all 8 parallel microfluidic channels are shown in a Zoomed-out view. (B) Again a 2-channel Zoom-in, taken from panel (A). (C) shows that within the channels, the design is an interweaving square lattice and hexagonal lattice regions to determine if this design will improve sample (cells or nuclei) loading over purely square lattice or purely hexagonal lattice micropillar arrays. (D), (E), and (F) show even more Zoomed-in cut-outs of what the alternating square versus hexagonal lattice regions look like up close.

FIG. 29A-B. An exemplary microfluidic device. (A) Flow direction goes from up-to-down, and all 8 parallel microfluidic channels are shown in a Zoomed-out view. (B) The design is to truncate/shorten the overall micropillar array region, where the micropillar array is shorter in length than other designs presented herein (e.g., FIG. 25 or FIG. 26), and can be adjusted to be any desired length overall, similar to how any other dimensions of the channel (width, height) or the micropillars (height, diameter, shape, spacing, etc.) can be freely adjusted as desired. The larger and more spaced out pillars downstream of the micropillar array region (near the outlet port; lower half of panel (B)) are support pillars purely there for structural purposes, and as well as other features on the design, can be changed as however desired.

FIG. 30A-D. An exemplary microfluidic device. (A) Flow direction goes from up-to-down, and all 8 parallel microfluidic channels are shown in a Zoomed-out view. (B) The design is to repeat the micropillar array regions separated by a distance in support structures, where the micropillar array can be either Square Lattice, Hexagonal Lattice, or a combination thereof, and can be at any gradient of spacings either Smooth or Stepped, and the repeated micropillar regions can be identical but can also differ completely. (C) Zoomed-in of a single channel with alternating micropillar versus empty/support structure regions. (D) Further Zoomed-in up close example of two micropillar array regions separated by a empty/support structure region. In this design, as with all other designs of all microfluidic channels presented herein, support structures can range in spacing, size, shape, and periodicity.

FIG. 31A-C. (A) Flow direction goes from up-to-down, and all 8 parallel microfluidic channels are shown in a Zoomed-out view. In this figure, as in FIG. 30, repeated regions of micropillar arrays are designed, but now with 3 regions of micropillar arrays rather than 2. Again, each micropillar array region is separated by a spacing of empty/support structure region. (B) and (C) are further Zoomed-in screenshots of the CAD drawings to better depict the differing regions. Important to note that the spacing for the 3 micropillar regions in (B) can be same or different, e.g., increasing in density of micropillars/decreasing in pillar-to-pillar distance, or any other desired variation or alternative spacing.

### DETAILED DESCRIPTION

The present disclosure relates to a system, method, and device for extracting genomic DNA from cell samples with tunable size selection. Each of the following patents and publications is incorporated by reference herein in their entirety: U.S. Pat. No. 10,947,528, Craighead et al. Microfluidic device for extracting, isolating, and analyzing DNA from cells; U.S. Pat. No. 9,926,552, Craighead et al. Microfluidic device for extracting, isolating, and analyzing DNA from cells; U.S. Pat. No. 9,856,513, Cerf et al. Methods and arrays for controlled manipulation of DNA and chromatin fragments for genetic and epigenetic analysis; Benítez, J. J., Topolancik, J., Tian, H. C., Wallin, C. B., Latulippe, D. R., Szeto, K., Murphy, P. J., Cipriany, B. R., Levy, S. L., Soloway, P. D., et al. (2012). Microfluidic extraction, stretching and analysis of human chromosomal DNA from single cells. Lab on a Chip 12, 4848; Tian, H. C., Benitez, J. J., and Craighead, H. G. (2018). Single cell on-chip whole genome amplification via micropillar arrays for reduced amplification bias. PLOS ONE 13, e0191520; Reinholt, S. & Craighead, H. G. (2018) Microfluidic Device for Aptamer-Based Cancer Cell Capture and Genetic Mutation Detection. Anal Chem. 90 2601-2608. doi: 10.1021/acs.analchem.7b04120; and H. W. Hou et al. Microfluidic Devices for Blood Fractionation. Micromachines 2011, 2 (3), 319-343; https://doi.org/10.3390/mi2030319.

### Definitions

The articles “a” and “an” are used herein to refer to one or to more than one (i.e. to at least one) of the grammatical object of the article. By way of example, “an element” means one element or more than one element.

Unless specifically stated or obvious from context, as used herein, the term “about” is understood as within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

All numerical ranges provided herein are understood to be shorthand for all of the decimal and fractional values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9 and all intervening fractional values between the aforementioned integers such as, for example, ½, ⅓, ¼, ⅕, ⅙, ⅛, and 1/9, and all multiples of the aforementioned values. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the term “sample” will be understood to encompass any fluid, solution or mixture, either isolated or detected as a constituent of a more complex mixture, or synthesized from precursor species. The term “sample” encompasses any biologically-derived sample or biological sample, including but not limited to blood, plasma, serum, lymph, saliva, tears, cerebrospinal fluid, urine, sweat, plant or vegetable extracts, semen, in vitro cell culture, tissue homogenates (e.g., animal tissue homogenate, plant tissue homogenate, etc.), a solution comprising dissociated cells or cell nuclei, and ascites fluid. The sample may or may not be native. For example, the sample may comprise acellular species, e.g., broken cells, nucleus. In addition, the sample may have been fixed or treated otherwise. In some embodiments, a sample or a biological sample comprises at least one cell, at least one cell nucleus, and/or at least a portion of gDNA (partially isolated or fully isolated).

The device of the present disclosure may be variable in size. The technology of isolating gDNA or fragments there of is not limited by the size of the device. Thus, the dimensions provided herein are merely for practical purposes, and the dimensions of the device can be bigger or smaller than indicated.

In some embodiments, the length or the width of the device may be equal to or less than about 450 millimeters.

In some embodiments, the height of the device or the height of the internal channel within the device may be in the range of 1 μm to 200 μm. In some embodiments, said height may be at least about 1, 10, 20, 30, 40, 50, 60, 70, 80, 90, 100, 110, 120, 130, 140, 150, 160, 170, 180, 190, or 200 μm. In some embodiments, said height may be in the range of 15 μm to 35 μm.

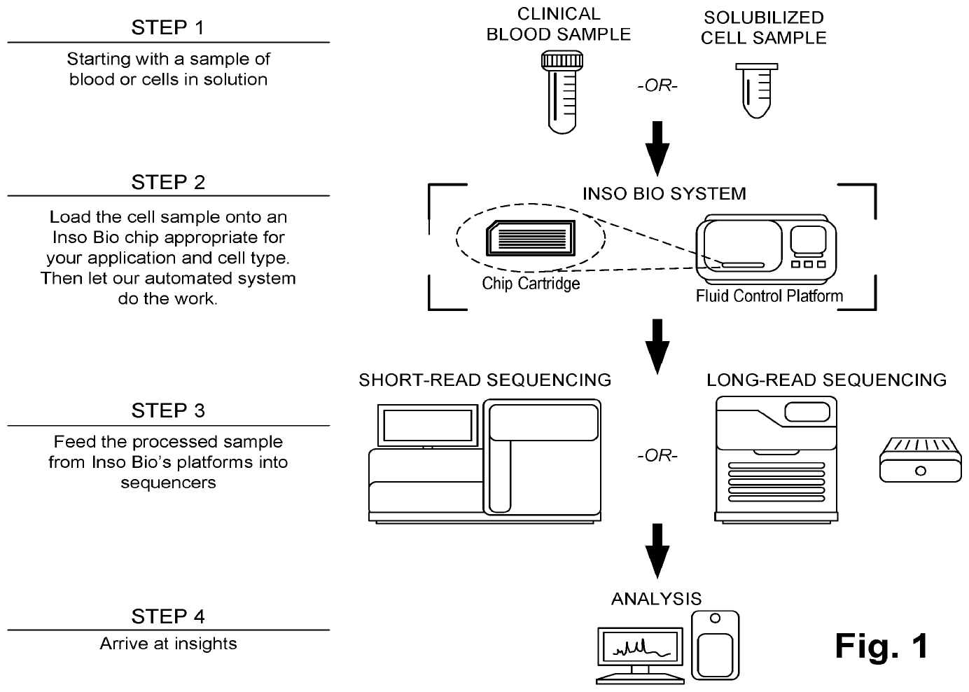

This invention is based on methods for extracting and isolating genomic DNA (gDNA) from samples containing cells. FIG. 1 depicts an exemplary workflow of a disclosed system.

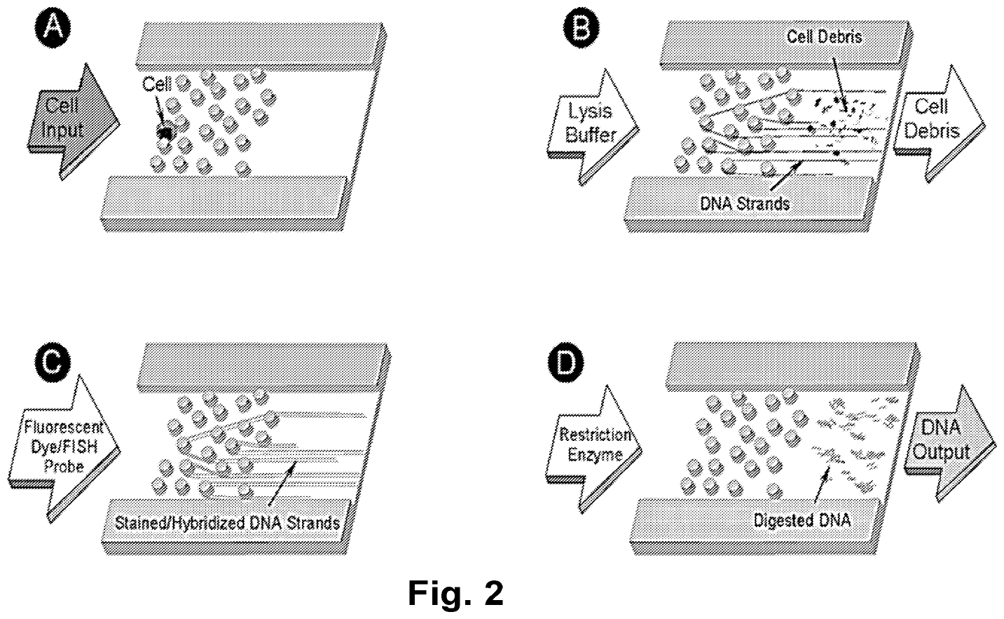

Shown in FIG. 2, an exemplary method begins with the introduction of a sample (e.g., whole blood, or cells in suspension) into a microfluidic device containing one or more microfluidic channels. This can be performed manually via pipetting or any other means such as utilizing a syringe pump, a peristaltic pump, or other means of controllably driving fluid.

As the samples are introduced into the microfluidic channels, a gentle pressure applied to the fluid pushes the cell solution through the channel towards an output reservoir. During this process, the cell solution passes through a region of the channel containing an array of micro pillars that alter the flow of fluid in the channels.

These micro pillars can have any shape or form. For example, the head of the micropillars and/or tip of the micropillars can comprise a shape of circles (thereby forming a cylinderous micropillar), ovals, squares, triangles, rectangles, cross shapes, hexagonals, diamonds, polygons, a dome (in 3-dimension), a pyramid, or any combination(s) thereof.

The micropillars can be arranged at variable spacing dimensions, depending on the size of the target cells of interest. Most animal cells (e.g., mammalian cells) are between 10 μm to 100 μm in diameter. HeLa cells are normally 10˜ 40 μm in diameter depending on the culture conditions. Red blood cells, one of the smallest human cells, have a diameter of less than ˜8 μm. Muscle fiber cells and neurons on the other hand can be extremely long. A mammalian cell nucleus (e.g., a human cell nucleus) can be ˜10 μm in diameter. Plant cells tend to be larger than animal cells but can still be between 10 μm to 100 μm in diameter. Fungus cells can be between 2 μm to 10 μm in diameter. A protist can be between 1 μm to 3 millimeters in diameter or length. Budding yeast Saccharomyces cerevisiae is ˜4 μm in diameter. The average diameter of spherical bacteria is 0.5 μm-2.0 μm. For rod-shaped or filamentous bacteria, length is 1 μm-10 μm and diameter is 0.25 μm-1 μm. Cell sizes and nucleus sizes are well known in the art. A cell or cell nucleus can be from any organism including an animal (a mouse, a dog, a cat, a human, a cow, etc.), a fungus, a protist, a bacteria, or a plant.

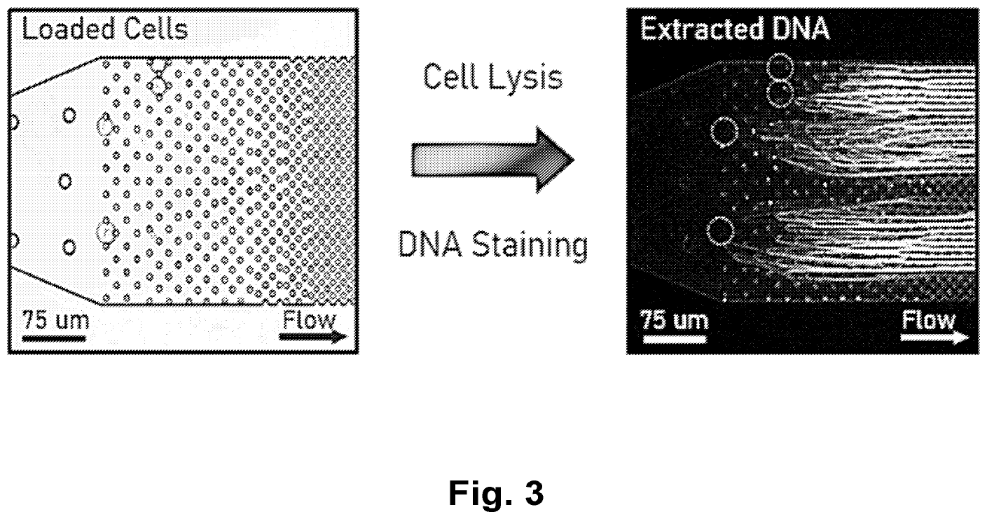

The next step in the process is to perform a brief wash step with a suitable buffer (e.g., phosphate buffered saline), which acts to remove any unwanted matter from the buffer and helps wash out any loose particles that might be stuck in the channel. Next, the genomic DNA (gDNA) of the trapped cells is released by flowing a lysis buffer into or through the device. Upon cellular lysis, the genomic DNA is released from the nucleus and the relatively long gDNA molecules become caught and retained on the micro pillars, while all other cellular components flow to the output reservoir, thus isolating the gDNA from all other cellular components. Another wash step is then performed to remove the lysis buffer from the channel. This is shown in FIG. 3.

As cells traverse through the array, target cells become trapped in the array due to the restriction of the pillar spacing, while all particles in the solution of sizes smaller than the restrictive pillar spacings will pass through unimpeded towards the output reservoir. For example, in a whole blood sample, the larger cells such as leukocytes will become trapped between the micro pillars due the restrictive micro pillar spacing, while smaller cells (e.g., red blood cells, platelets, bacteria) and other acellular matter (e.g., virus particles, proteins, lipids, RNA) pass through the micro pillar array unimpeded to an output reservoir.

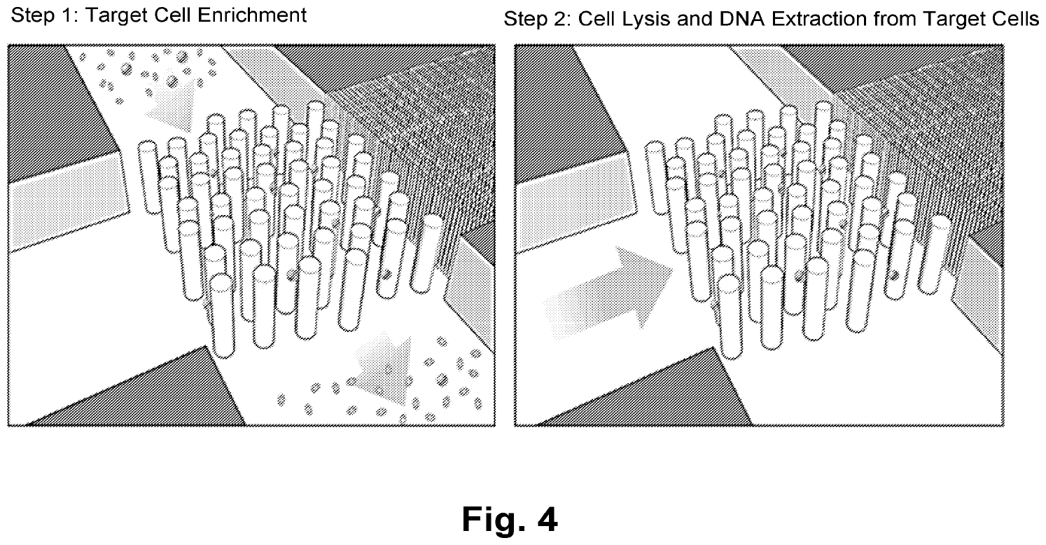

Alternatively, if an affinity selection approach is taken, the channel can be pre-treated to functionalize the channel with an antibody or antibodies (or other molecules conferring specific affinity such as aptamers), and the pillar spacing can be made less restrictive (e.g., larger than necessary to entrap the cells) to be able to select between cells of similar size expressing different surface markers. With this approach, only cells expressing a certain marker or sets of markers will be trapped in the pillar array, while other cells will pass through the array. A combination of size selection and affinity selection can also be employed. Such combination can be in any sequence or configuration. In some embodiments, an array of micropillars for affinity capture can be placed closer to or farther from inlet port relative to an array of micropillars for size selection. In other embodiments, a mixture of micropillars for affinity capture and micropillars for size selection can be disposed within the same area in the channel. In still other embodiments, different arrays of micropillars (e.g., for affinity capture vs. size selection) can be disposed in different channels (e.g., see the perpendicular or bifurcating channel below, e.g., FIG. 4).

This strategy of channel surface functionalization can be utilized to capture cells of interest from within a sample of mixed biological components. Shown in FIG. 4, an example application would be to utilize channel functionalization on larger structures at the intersection of two microfluidic channels in a perpendicular orientation. While the secondary channel that is in the perpendicular direction is shown as an example in FIG. 4, the secondary channel that crosses the first channel can be at any angle with respect to the first channel. The larger structures can be functionalized with antibodies, DNA/RNA aptamers, or any other molecule conferring the capability of selectively targeting and arresting a specific type of cells while allowing all other cells to flow through unhindered. The captured target-cells can then be lysed towards a pillar array region in the perpendicular channel. This enables the ability to selectively capture and extract DNA from cells of interest, and to then collect and analyze the target-cells' genetic material while other cells or debris can be separated from this collection.

To recover the gDNA, in the flowing system, the entangled DNA is cut into smaller fragments by using enzymes that cleave double stranded DNA. The smaller fragments of DNA escape more easily from the pillars and flow towards the output reservoir. The gDNA can then be removed from the device for downstream processing or undergo further processing such as heat inactivation of enzymes, buffer exchange, sample concentration or library preparation for sequencing platforms on a second stage of the device. The gDNA is then compatible for any analytical processes such as amplification, genetic identification, or sequencing. The present disclosure encompasses various means to cut entangled DNA into smaller fragments, e.g., endonuclease digestion (e.g., restriction enzymes), sonication, high intensity light, altering flow condition, any other methods of cutting gDNA into smaller fragments that are known in the art, or any combination thereof (see below for further discussions).

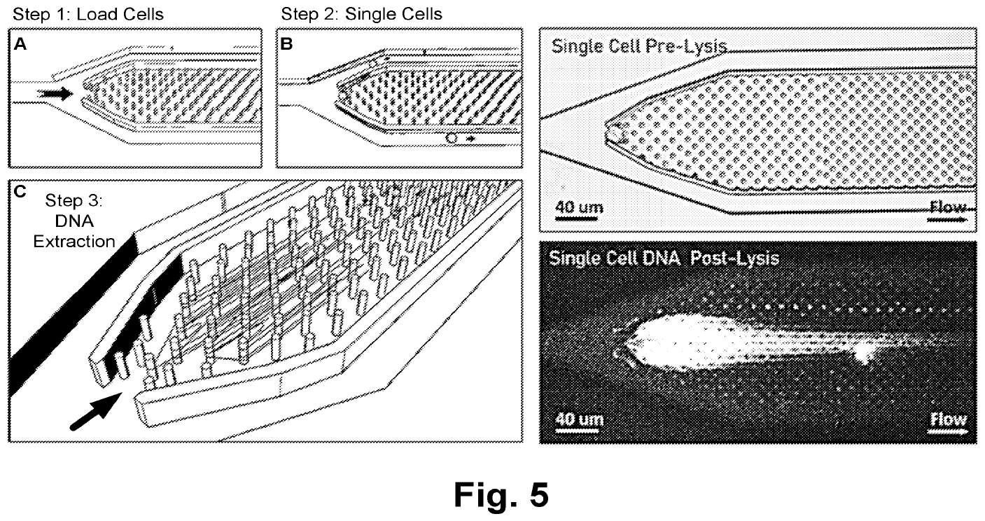

The scalability of the device can be brought down to single cell processing, where functionality and performance of the micropillar array technology is preserved. FIG. 5 shows a single cell workflow, which is very similar to the multi-cell workflow described in FIG. 2. Here, built-in walls enclosing the micropillar array region allow only single cells to be captured at the apex of the micropillar array. Once a cell is caught, access to the micropillar array is occluded for additional cells, and any additional cells coming into the channel are redirected via ‘gutter’ bifurcation channels to downstream waste channels or further connected to additional single cell channels. Upon cell lysis, only single cell gDNA is collected in each micropillar array and allows for further single cell analysis once the DNA is cleaved and collected.

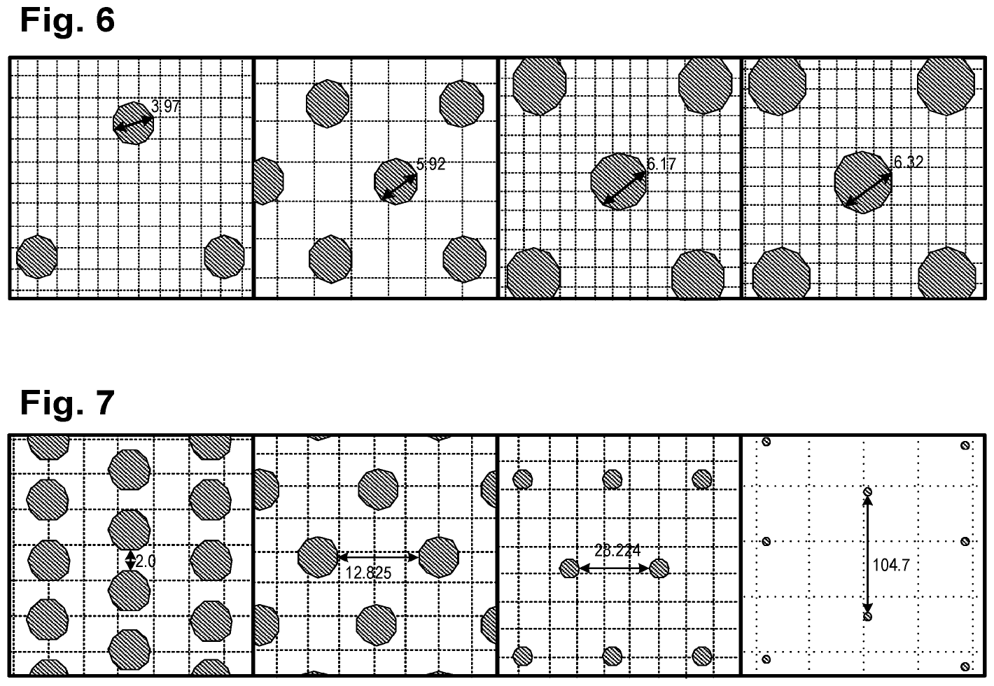

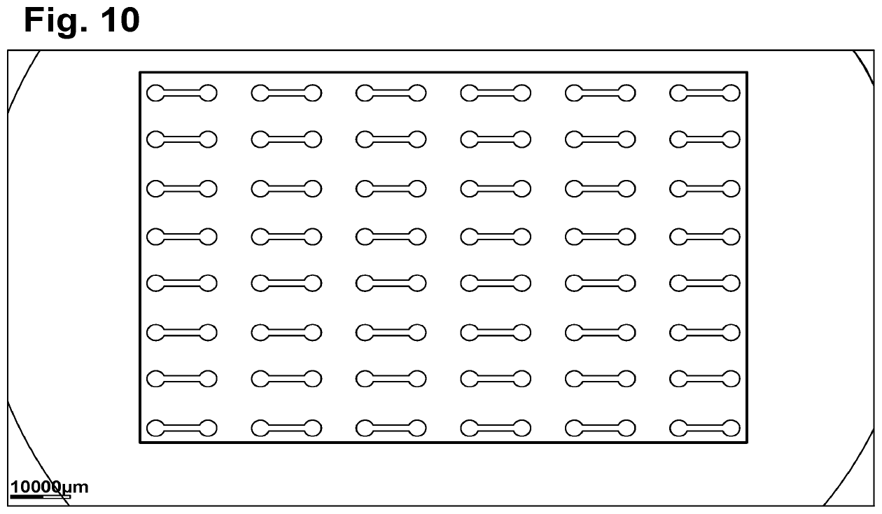

FIG. 2 through FIG. 5 show a microfluidic device that consists of an array of pillars fabricated in PDMS by lithographic process. The PDMS structures are capped with a glass plate to form fluidic channels that incorporate microstructures to capture selected cells and subsequently capture and select DNA of selected fragment lengths. The micropillar array can be configured as desired in all aspects of dimension, and this can be specifically tailored for small or large samples. FIG. 6 through FIG. 11 show designs of various devices.

Functionality of the microfluidic system can be preserved through a wide range of channel configurations and dimensions. Within the microchannel, this configurability includes the size, shape, density, and spacing of the microstructures that are responsible for the capture of cells and extraction and retention of genomic DNA during cell-lysis. For the overall chip dimensions, the height, width, and length of the channel can be changed while simultaneously preserving system functionality. This includes not only scaling the total size and capacity of the chip, but also configuring the chip into various formats such as formats suitable for single cell processing, formats suitable for large sample volume processing, and 96-well or microwell plate formats that can allow additional levels of integration to other sample preparation and/or analytical systems that operate within a microwell plate configuration. These alternative microwell plate systems include but are not limited to other robotic sample preparation platforms, flow cytometer platforms, plate-readers, etc.

The disclosures here demonstrate and contemplate several systems, devices and methods for preparing and collecting prescribed fragments sizes of DNA from selected cells. These result in improvements for DNA preparation for sequencing and other analyses.

### System

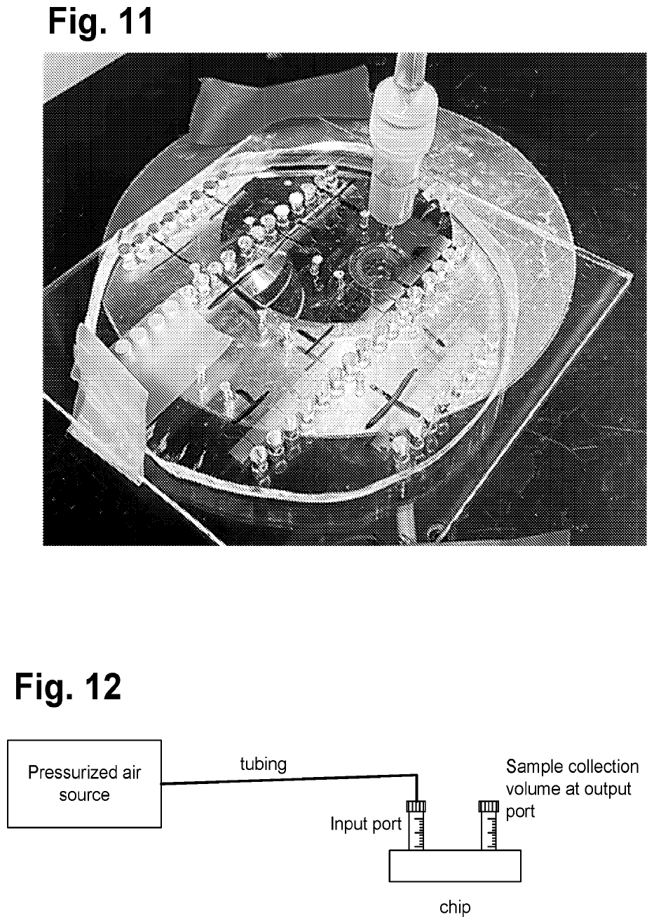

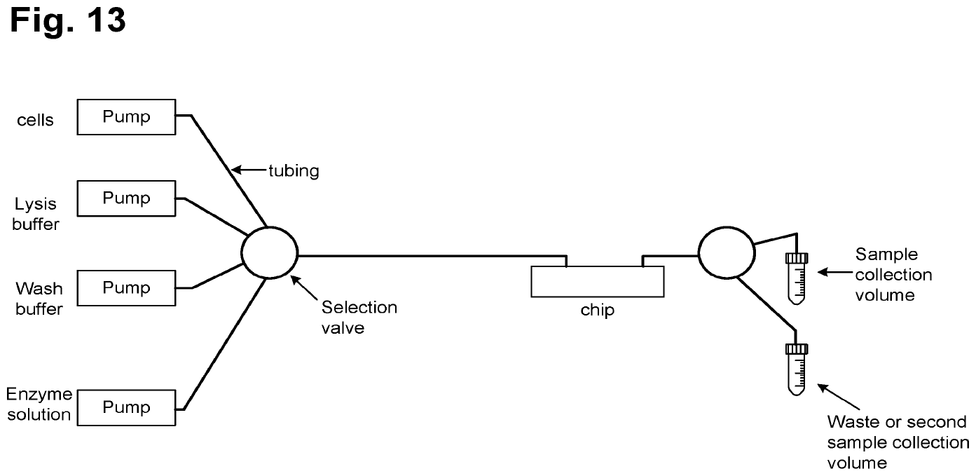

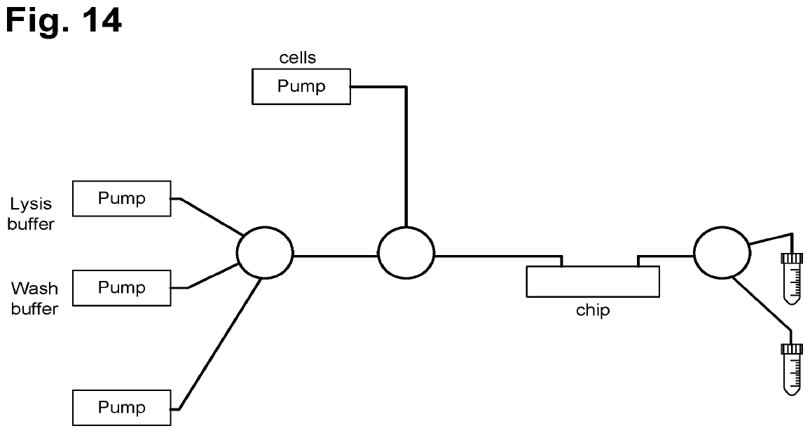

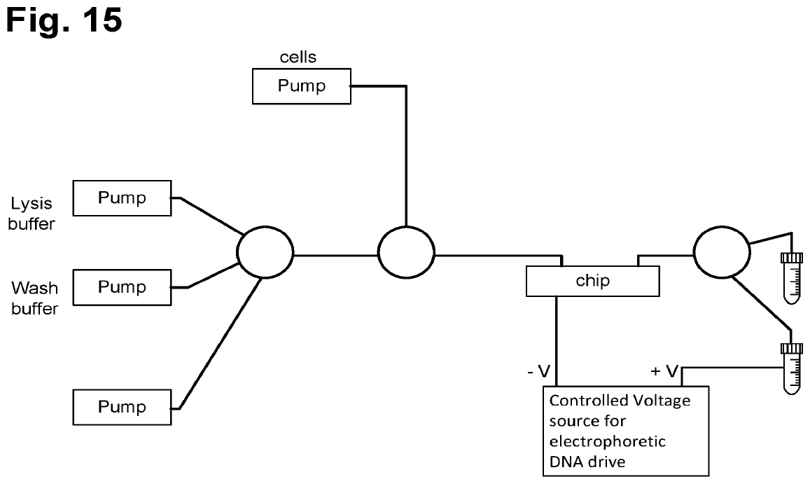

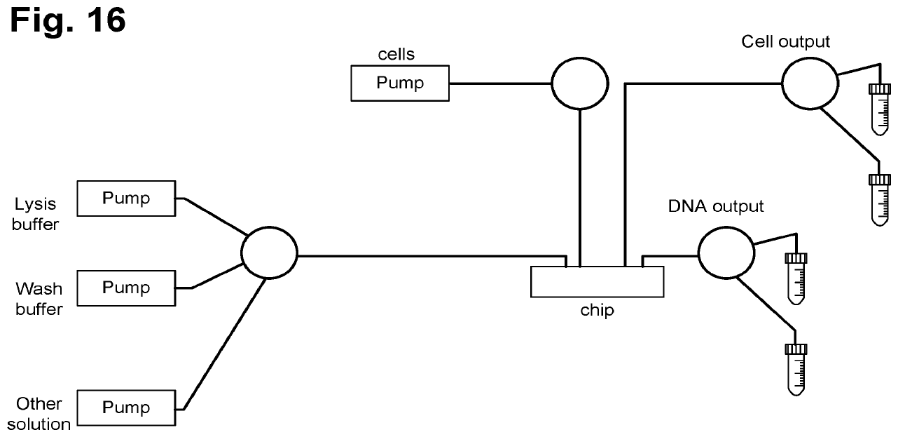

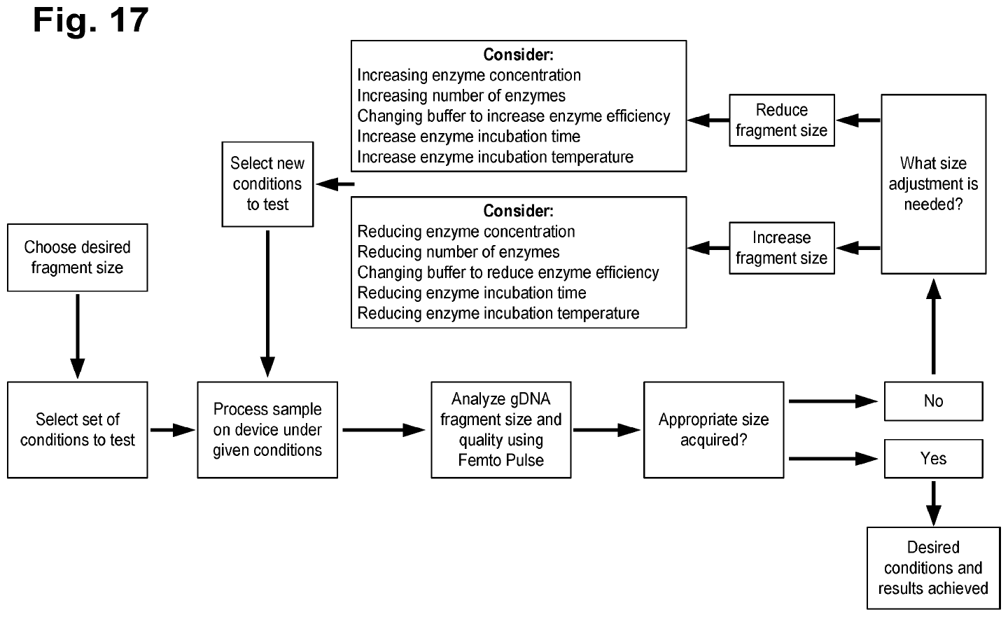

As shown in FIG. 11, the disclosed system comprises a mirofluidic “chip” device and a system for delivering the sample to the microfluidic chip device, controlling the sequential flow of different reagents through the microfluidic chip to perform the cell and DNA processing.

One fluid system that has been frequently used is simple in which the fluid samples and reagents are pipetted into an inlet port with a volume of about 10 microliters to about 50 mL. In some embodiments, the volume is at least about 0.01, 0.02, 0.03, 0.04, 0.05, 0.06, 0.07, 0.08, 0.09, 0.1, 0.12, 0.14, 0.16, 0.18, 0.2, 0.22, 0.24, 0.26, 0.28, 0.3, 0.32, 0.34, 0.36, 0.38, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mL; or any range between these values, that is integrated into the PDMS part of the fluidic chip. FIG. 6 shows an image of a typical microfluidic device with the inlet and output ports attached. The fluid is driven through the microfluidic chip device by applying pneumatic pressure of a few PSI from compressed air. The air pressure is applied by inserting a tube connected to a controlled pressure source into the inlet port of the device for a duration necessary to drive the desired amount of fluid. The tube is removed after driving the flow to allow pipetting the next liquid or reagent needed for the process into the inlet port followed by another pressure driven step.

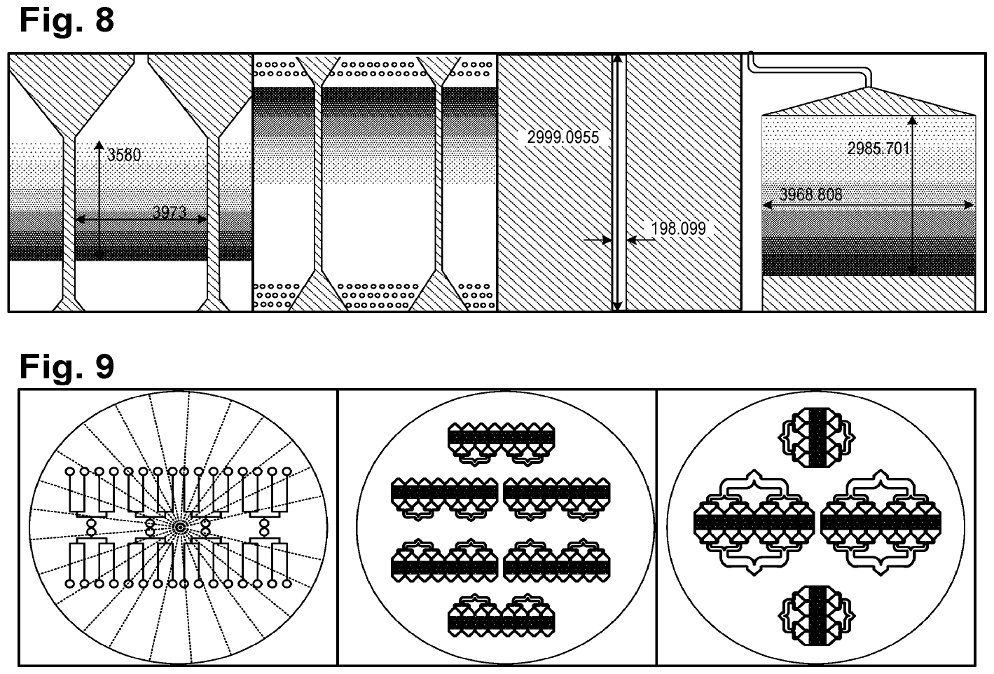

The present disclosure encompasses other systems that do not require attaching and detaching a tube to apply pressure to drive the required fluids. FIG. 8 shows a schematic of such a system in which the fluids can be driven by any pump type, such as pressurized air source (e.g., compressed air), controlled pressure source, controlled pneumatic pressure source, pressure driven pump, syringe pump, vacuum pump, or a peristaltic pump with a selecting valve to choose the fluid needed for the required process step. The flow rate and volume can be measured and controlled by standard processes.

### Methods

The method for extracting size selected DNA fragments involves using specific combination of cocktails of DNA cleaving enzymes, applied flow pressure, incubation time, and temperature to produce gDNA fragments of specific fragment lengths.

Any sample or solution (including but not limited to digestion buffer, wash buffer, elution buffer, lysis buffer, etc.) can be loaded into the microfluidic device using an applied pressure (e.g., measured in positive psi or flow rate) or vacuum pressure (e.g., measured in negative psi or flow rate).

In some embodiments, the applied pressure or vacuum pressure may be at least about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 psi; or any range between these values. Here, the psi in reference to applied pressure indicates positive psi, whereas the psi in reference to vacuum pressure indicates negative psi.

In some embodiments, the flow rate may be at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 μl/min. In some embodiments, the flow rate ranges from 0.1 μl/min to 1000 μl/min. In some embodiments, the flow rate ranges from about 0.1 μl/min to about 500 μl/min.

Loading of a Sample Comprising Cells into a Microfluidic Device

A sample comprising cells in suspension (e.g. dissociated animal cells (e.g., mammalian cells), whole blood) of volume ranging from at least about 10 microliters to about 50 mL (e.g., at least about 0.01, 0.02, 0.03, 0.04, 0.05, 0.06, 0.07, 0.08, 0.09, 0.1, 0.12, 0.14, 0.16, 0.18, 0.2, 0.22, 0.24, 0.26, 0.28, 0.3, 0.32, 0.34, 0.36, 0.38, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mL; or any range between these values) is loaded into a microfluidic device containing one or more channels. Each channel consists of an inlet port, an array of micro pillars, an output reservoir, and a second stage for further processing including sample heating, buffer exchange, sample concentration and library preparation.

Loading the sample onto the device is achieved either by pipetting the solution manually into an apparatus interfacing with the microfluidic channel that can hold the sample volume, or through pressurized air source (e.g., compressed air), controlled pressure source, controlled pneumatic pressure source, pressure driven pump, syringe pump, vacuum pump, or a peristaltic pump or similar device. The sample containing cells is then driven through the channel using an applied pressure to the apparatus (0.001 to 50 psi) or via pump system at defined rates.

The sample is then allowed to flow into the device until the desired volume has been driven through the device (1 second up to hours). During this time, the sample passes through an array of micro pillars. The pillar size, spacing, and length of the array can be customized to create a pillar array tailored for specific cell types with specific spacing between the pillars (in certain embodiments, minimum of 1 micrometer). As the cells encounter the array, any cell that is larger than the pillar spacings will become physically trapped and unable to continue passing through the array and channel. Any cell or other particle that is smaller than the pillar spacings can flow through the channel unimpeded, towards the output reservoir.

For example, for a sample comprising whole blood, an array with a pillar spacing gradient ranging from 100 to 8 micrometers will trap leukocytes or circulating tumor cells in the array while letting all other components of the whole blood (e.g. erythrocytes, thrombocytes, albumin, bacteria, viruses etc.) pass through the pillars unimpeded due to their smaller size. These smaller components will continue to flow through the channel and pool in the output reservoir, where they can be removed as waste or can be used for further off-device analyses.

If affinity selection is desired, the channel and pillars can be functionalized with specific antibodies (or aptamers or any affinity conferring proteins, solutions, molecules, or nucleic acids). For example, if mature T-cells were desired to be isolated from whole blood, the channel and array could be treated to be functionalized with anti-CD3, a specific marker for mature T-cells, and the pillars could be defined to physically allow all leukocytes to traverse through the pillars. Therefore, CD3− cells would pass through, while CD3+ would be captured through affinity. Antibodies that bind various cell surface markers are available commercially. For example, antibodies that bind CD3 can be purchased from vendors such as R&D systems (representative catalog #MAB100, MAB100R, FAB100A, etc.), Abcam (representative catalog #ab11089, ab5690, ab52959, etc.), ThermoFisher Scientific (representative catalog #14-0037-82, 16-0037-81, 48-0037-42, 16-0038-81, 13-0037-82, etc.), and BioLegend (representative catalog #317319, 317301, 344801, etc.). Labeled antibodies (e.g., biotin-labeled antibodies) are also available from the commercial vendors, which then can be easily attached to micropillars that are coated with streptavidin. Finally, the channel containing the functionalized micropillars (e.g., with an antibody or an aptamer) can be exposed to a solution comprising at least one cell or cell nucleus at an applied pressure of (about 0.001 to about 50 psi; or at least about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 psi; or any range between these values) or at a defined flow rate for 1 minute up to hours or days. In some embodiments, the total volume of the solution that flows into the device may range from at least about 10 μl to about 100 ml (or the range between these values). In some embodiments, the flow may be stopped to allow incubation of the cells with the functionalized micropillars as to maximize the binding. Similar to the cell binding, the trapped/bound cells can be washed by changing the solution to a wash buffer (e.g., phosphate buffered saline, pH 7-7.9) using the same pressure, flow rate, or amount of buffer indicated above.

Lysis of Captured Cells and Trapping of gDNA

To release the gDNA contents from the cells trapped between the pillars, a lysis buffer suitable for lysing the nuclear membrane of the cells (e.g., 1% SDS in PBS pH 7-8, or 4M guanidinium isothiocyanate) is added to the device and allowed to flow over the cells for about 0.001-about 120 minutes with an applied pressure (about 0.001 to about 50 psi—e.g., 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 psi). The lysis buffer composition may be altered such that native histone complexes are retained upon lysis. Thus, the amount of proteins bound to the recovered genomic DNA can be controlled and modulated by the types of lysis buffer and/or wash buffer (e.g., comprising a harsher or a milder denaturant, etc.). During lysis, all cellular contents are released from the cell or cells, but the genomic DNA becomes tangled and caught on the micro pillars while all other contents (lipids, proteins, RNA, etc.) flow past the pillars and towards the output reservoir where they can be removed from the device as waste or for further analysis. The pillars retain nearly 100% of the expected genomic DNA contained in the cell or cells. Phosphate buffered saline is then added to the device to wash out the lysis buffer for about 0.1 seconds to about 120 minutes under a pressure of about 0.001 to about 50 psi. In some embodiments, the pressure is at least about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 psi. Under these conditions, the trapped gDNA remains on the pillars for several days.

Tunable Shearing and Release of gDNA

To release and recover the gDNA from the micro pillars, the following exemplary methods may be used:
1. Enzymatic cleavage
2. Altering flow conditions
3. Sonication
4. Light method (e.g., High intensity light)
5. Nebulization
6. Mechanical method/Melting or collapsing pillars
7. Any combination of 1-5
   A person of ordinary skill in the art understands that in addition to the above methods, any known methods of cutting/shearing gDNA that are known in the art can be combined with the devices, systems, and/or methods of the present disclosure.

### Enzymatic Methods

Using different enzymatic processes and conditions, the size fragments of the gDNA recovered can be tuned to specific size ranges to meet the optimal conditions for the desired downstream analytical applications. This can be achieved utilizing any enzyme capable of performing nuclease cleavage of double stranded DNA.

Such examples include, but are not limited to: restriction enzymes (e.g., type-I restriction enzymes (restriction-and-modification enzymes that cut DNA far from their recognition sequences), type-II restriction enzymes (blunt or overhang cutters), type-III restriction enzymes (cleave outside of their recognition sequences and require two such sequences in opposite orientations within the same DNA molecule to accomplish cleavage), type-IV restriction enzymes (enzymes that recognize/cleave modified, e.g., methylated DNA)), homing endonucleases, transposases, type-II CRISPR-Cas9 proteins or other commercially available enzymes that cut dsDNA (e.g. dsDNA Fragmentase from New England Biolabs (representative catalog #: M0348S, M0348L)). The size of the gDNA recovered from the device can be selectively tuned based on several conditions such as: type and number of enzymes used, the concentration of enzyme or enzymes, buffer composition, incubation temperature, incubation time, and flow conditions (psi and time at specific psi).

General Outline of Enzymatic Method with Restriction Endonucleases

To recover gDNA fragments within a specific fragment size range, a reaction mixture is prepared containing the following:
one or more type II restriction enzymes and or homing endonuclease (1×10⁻⁴ to 5 U/microliter final)
in a buffer containing
   Tris-HCl (0-500 mM)
   NaCl (0-500 mM)
   MgCl2 (0-100 mM)
   Potassium acetate (0-500 mM)
   Tris acetate (0-500 mM)
   DTT (0-10 mM)

The buffer is introduced to the microfluidic channel through the apparatus by pipetting or a syringe pump. The device is set to a controlled reaction temperature (10 to 70) C°. The reaction is allowed to proceed under a flow pressure of 0.01-50 psi, preferably for 1-60 minutes, and then 1-30 minutes at 0.5-50 psi. Notably, in certain situations, a higher flow rate during the DNA release (relative to the flow rate used during the enzyme incubation) results in higher yield in the recovered gDNA fragments. In some embodiments, the higher flow rate during the DNA release (“blast”) may be applied using the pressure of at least about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 psi. In some embodiments, the blast may be applied at a flow rate in the range of at least about 0.1 μl/min to about 1000 μl/min (or any range between these values). In some embodiments, the blast may be applied for a duration of about a fraction of a second (e.g., millisecond range). In some embodiments, the blast may be applied for a duration of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 seconds. In some embodiments, the blast may be applied for a duration of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes. In preferred embodiments, the blast is applied for a duration in the range of at least about 0.1 second to 10 minutes.

In general, the following principles allow the user to direct the tunability of the fragment size recovered. The changes can be made individually, or in various combinations to produce different fragment sizes.
1. For Higher fragment sizes
   1. reduce total enzyme concentration
   2. reduce number of different enzymes
   3. change buffer to reduce enzyme efficiency
   4. reduce digestion time
   5. reduce digestion temperature
   6. alter flow conditions
2. For Lower fragment sizes
   1. increase total enzyme concentration
   2. increase number of different enzymes
   3. change buffer to increase enzyme efficiency
   4. increase digestion time
   5. increase digestion temperature
   6. alter flow conditions

### Other Enzymatic and Non-Enzymatic Approaches for Fragmenting DNA

The following other enzymatic approaches may also be used to shear and release the gDNA from the pillars at defined sizes.
Type II CRISPR-Cas9
Transposases
Fragmentase
Tagmentation approaches, such as the bacterial transposase Tn5, including ones that in addition to fragmenting the DNA, leave the DNA altered (such as barcoding the ends of the fragmented DNA with nucleotides or other adaptors used in downstream methods)
Commercially available enzymes that fragment double stranded DNA

Physical forces can also be used to fragment the DNA as an alternative to enzymatic cleaving. For example, increased liquid flow rate will create hydrodynamic forces on the immobilized gDNA causing the DNA to fragment. Ultrasonic agitating of fluid (e.g., sonication) is also known to cleave DNA and may be applied to the immobilized gDNA to fragment it. Ultra-violet light could be focused on the device to fragment/release the gDNA alone or in combination with the other aforementioned processes.

Accordingly, the devices, systems, and/or methods of the present disclosure may further comprise at least one agent and/or at least one instrument that facilitate (a) cutting gDNA into fragments and/or (b) modifying the gDNA fragments or the proteins associated with said fragments.

In some embodiments, the at least one agent comprises at least one enzyme. In some embodiments, the at least one enzyme may comprise a DNA-cleaving enzyme, a DNA-modifying enzyme (e.g., DNA methyltransferase, terminal transferase that can label the DNA fragments, T4 DNA ligase that can ligate a PCR primer to the DNA fragments for amplification, DNA polymerase (e.g., for PCR amplification; e.g., Taq polymerase, Pfu polymerase, etc.), and/or an enzyme that modifies DNA-associated proteins (e.g., histones, transcription factors, etc.) (e.g., histone methyltransferases, demethylases, acetyltransferases, deacetylase, kinases, phosphatases, ubiquitin ligases, deubiquitinating enzymes, O-GlcNAc transferase (OGT), O-GlcNAcase (OGA), E3 SUMO ligases, SUMO-specific proteases, proteases, Poly-ADP ribose polymerase, (Adp-ribosyl) hydrolases ARH1 & ARH3, Protein arginine deiminase 4 (PAD4), Fpr4 (proline isomerization)).

In some embodiments, the at least one instrument comprises an ultrasonic liquid processor (e.g., a sonicator).

In some embodiments, the at least one instrument comprises a light source for high intensity light that fragments gDNA. In some embodiments, the light source for high intensity light emits/produce UV light, X-rays, gamma rays, and/or ionizing radiation. In preferred embodiments, the light source emits UV light.

In some embodiments, the at least one instrument comprises a nebulizer. It is art-recognized that nebulization results in DNA fragmentation (see e.g., Lentz et al. (2005) J Aerosol Sci 36:973-990; Sambrook and Russell (2006) CSH Protoc pdb.prot4536; each of which is incorporated herein by reference).

In some embodiments, the at least one instrument comprises an instrument that creates a hydrodynamic force that can fragment gDNA (e.g., Point-sink Shearer (PtS), recirculating point-sink flow system, bubbling system, and/or any instrument described in e.g., Yew and Davison (1968); Thorstenson et al. (1998) Genome Res 8:848-855; Shui et al. (2013) Rsc Adv 3:13115-13118; Oefner et al. (1996) Nucleic Acids Res 24:3879-3886; Nesterova et al. (2012) Lab Chip 12:1044-1047; Joneja and Huang (2009) Biotechniques 46:553-556; Shui et al. (2011) Nanotechnology 22:494013; Li et al. (2017) Scientific Reports 7:40745; each of which is incorporated herein by reference.

### Light Method

In certain aspect, a non-enzymatic approach/method is used to cleave and release the gDNA from the micropillars. Such a method may comprise the use of directing light (potentially at a high intensity) directly onto or focused onto the regions of the chip cartridge (device) where the gDNA is captured/extracted. In this approach, one or more light sources can be applied directly above or below the channel at a defined angle or set of angles defined coordinates (or scanning an entire region). The light should be at minimum a strong enough level of intensity to cause double stranded breaks alone or in conjunction with a chemical agent (including but are not limited to a DNA binding agent or fluorescent dye); or to create multiple single-stranded DNA breaks that collectively would weaken the physical/mechanical properties of the gDNA to then allow the gDNA to be more easily released or freed from their tethering pillar in the micropillar array. This can be combined with the aide of increasing shear forces via increased fluid flow or change in internal channel pressure, or enzymatic cleavage, or any other method of cleaving mentioned.

The same principle utilized in the Light Method can also be extended to wavelengths beyond the visible spectrum of the human eye, including X-Rays, UV-Rays, or other wavelengths such as but not limited to radioactive wavelengths.

### Sonication Method

In certain aspect, a non-enzymatic approach to cleaving and releasing the gDNA from the micropillars comprises the use of sonication to shear the extracted gDNA and therefore enable its release from the micropillars, which can be done with or without constant or intermittent fluid flow through the microfluidic channels. In this use, one or more sonication sources may be embedded directly into the channel, seated directly above or beneath the channel, or the entire chip may be housed or placed into a sonication chamber or sonication solution/sonication bath. When sonication forces are applied either intermittently or consistently, the gDNA will be sheared. If fluid flow is then subsequently or simultaneously applied to the microfluidic channels, the sheared/sonicated DNA will be freed from the physical/mechanical entanglement forces that originally tethered the gDNA during extraction/cellular or nuclei lysis, and thus can be subsequently flowed recovered/retrieved/collected.

### Altering Flow Method

Flow rate can be manipulated several ways to affect the factors and methods used to release the gDNA or gDNA fragments from the pillars. For example, while using enzymes to cleave the gDNA, flow rate can be slowed down to allow for enzymatic interactions and processes with the DNA to occur more frequently or faster, resulting in more DNA cleaving. Conversely, flow rates could be sped-up to minimize enzyme-to-DNA interaction events. In another use, after enzymatic cleavage has weakened the physical/mechanical entanglement forces between the DNA and the micropillars, increasing the flow rate can induce higher shear forces imparted onto the DNA molecules, which would further untangle or shear the DNA from the micropillars and thereby act to “sweep/flush” the channel and help improve/maximize the recovery/yield of the DNA collected from the original cellular or nuclei sample input into the chip cartridge(s).

### Mechanical Method/Melting or Collapsing Pillars

To enhance the release and recovery/yield of extracted gDNA physically entangled within the micropillars without the use of any biochemical/enzymatic processing, some methods may include the use of micropillars that can be mechanically bent (no longer perpendicular to the flat channel), mechanically retracted (into one wall or cap layer of the chip cartridge/microfluidic channel), mechanically removed via peeling off of the layer attached to the micropillars, or chemically dissolved entirely or partially to allow the gDNA to slip or fall off of the micropillars under fluid flow, to be collected un-fragmented or minimally fragmented in the output port. The micropillars could be composed of a soft polymer (like PDMS) that contain paramagnetic particles that could then be manipulated or actuated to lean towards one direction with a single magnetic situated above the channel. Similarly, to dissolve the micropillars and thereby release the gDNA for collection, a solution could be flowed through the channel that dissolves partially or all of the micropillar material while being inert or minimally damaging to the gDNA within the channels.

Sizes of gDNA Fragments

The devices, systems, and methods of the present disclosure can recover intact genomic DNA, including a whole chromosome. Alternatively, the devices, systems, and methods of the present disclosure can also generate various sizes of gDNA fragments. For example, the size of gDNA fragments may be between at least about 1 kb to 500 kb; or any range between these values. In some embodiments, the size of gDNA fragments may be between at least about 1 kb to 500 kb; or any range between these values. In some embodiments, the size of gDNA fragments may be at least about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 kb; or any range between these values.

The term “size of gDNA fragments” does not mean that all fragments have identical size (e.g., length, molecular weight). Rather, it refers to the representative or average size of the gDNA fragments in the recovered solution. For example, in some embodiments, if the size of the gDNA fragments is at least about 5 kb, then the average size of at least about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the gDNA fragments in the recovered solution is at least about 5 kb.

In some embodiments, the size of the gDNA fragments represents the average size of at least about 50% of the gDNA fragments in the recovered solution.

In some embodiments, at least about 30%, 35%, 40%, 45%, 50%, 55%, 60%, 65%, 70%, 75%, or 80% of the total genomic DNA within the channel after cell/nucleus lysis comprises the desired fragment size+/−10 kb.

In some embodiments, at least about 50% of the total genomic DNA within the channel after cell/nucleus lysis comprises the desired fragment size+/−10 kb.

Recovery of Sample from Device

As the gDNA is cut during the shearing reaction, gDNA will be released from the pillars and flow towards the output reservoir. Upon completion of the reaction, the gDNA may then be processed in one of two ways.

(1) The first method involves removing the sheared gDNA from the output reservoir either by manual pipetting or through tubing whereby the sample is pumped into a collection tube off the device. The total output volume comprising the size selected gDNA may be at least about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.12, 0.14, 0.16, 0.18, 0.2, 0.22, 0.24, 0.26, 0.28, 0.3, 0.32, 0.34, 0.36, 0.38, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mL; or any range between these values. In preferred embodiments, the total output volume comprising the size selected gDNA may range from 1 microliter to 5 mL; or any range between these values. If enzymes were used, the sample is then incubated for a specific time at a specific temperature as indicated for the specific enzymes used (example: 15 minutes at 80° C.) to inactivate the enzymes present in the sample.

(2) The second approach involves a second, continuous stage of the channel. Upon completion of the shearing reaction, a valve downstream of the output reservoir is opened, which exposes the sample to an electrophoretic channel.

After enzymatic inactivation, the gDNA sample is then ready for downstream applications such as pulse-field gel electrophoresis, amplification and detection of specific loci, or sequencing.

In some embodiments, any sample or buffer (e.g., lysis buffer, wash buffer, elution buffer) may further comprise a blocking agent (e.g. BSA, ionic detergent, or another charged agent) and/or a lubricant (e.g., a polymer) to maximize the yield of the recovered gDNA fragments or the recovery of an intact chromosome or gDNA. Such blocking agent and/or a lubricant may ease the entanglement of the gDNA to the micropillars or surface of the channel as to avoid shearing the intact or large size gDNA by physical pulling. See below for further discussion.

### Mechanical & Electrical Micropillar Pertubations

Mechanical perturbations to the micropillars can be performed in a variety of manners to one or more of the micropillars within the micropillar array either simultaneously, in sequence, or at other intervals. Pertubations can include mechanical tilting or collapsing or the micropillars, mechanical or physical retraction of the micropillars, or by entirely removing the micropillars through peeling of the channel layer/wall that the micropillars are attached to. These micropillars can also be embedded with particles that can be magnetically or electrically engaged to alter shape or other mechanical or physical properties with the goal of enabling release of extracted genomic DNA without the need for DNA cleaving or fragmentation. Electrical perturbations can involve the application of electric charge or field to cause or induce electrically receptive components (wires, metallic anchors, etc.) within the micropillars to force shape changes to the overall micropillars.

### Chemical Micropillar Pertubations

Chemical perturbations can involve the partial or complete dissolving or degrading of the micropillar structural material via solutions flowed into the microfluidic channels. Chemical perturbations can also involve the damaging or degradation of particular portions of the micropillar in a manner as to allow the release of the micropillar from fixed or attached positions within the channels. This can also be accomplished by targeted perturbations to a specific subset of the micropillars within any number of micropillar arrays on a chip cartridge/within a channel, and can be selected for by the material of the select micropillars meant for DNA extraction and subsequent perturbation, but is not limited to such structures, and can be applied in concept towards support structures, walls, or other designed structures within the channel.

### Coating Micropillars for Slipping of DNA

One or more micropillars within the micropillar array or even entire channels can be coated with chemicals or materials that would cause DNA that is extracted and entangled within the micropillar array to slip off of the micropillars and therefore be recoverable/collected without the need of fragmentation. The molecule (such as PEG or PEO polymer, or even BSA or other biomolecules) can be adjusted as well as the incubation time within the channels, which would correlate to the percentage of the total internal surface of the channel or the micropillars to be coated, and/or would correlate to the thickness of the coating on the surface itself for such structures.

### Other Device Configuration Variations for the Disclosed Microfluidic Channels

In addition to the basic micropillar array layout in which variations exist for pillar shapes, pillar dimensions, and pillar-to-pillar spacing, tertiary structures can also be designed within the micropillar array to provide improved functionality during cell loading. Exemplary variations are described below.

### Additional Canals in the Micropillar Array

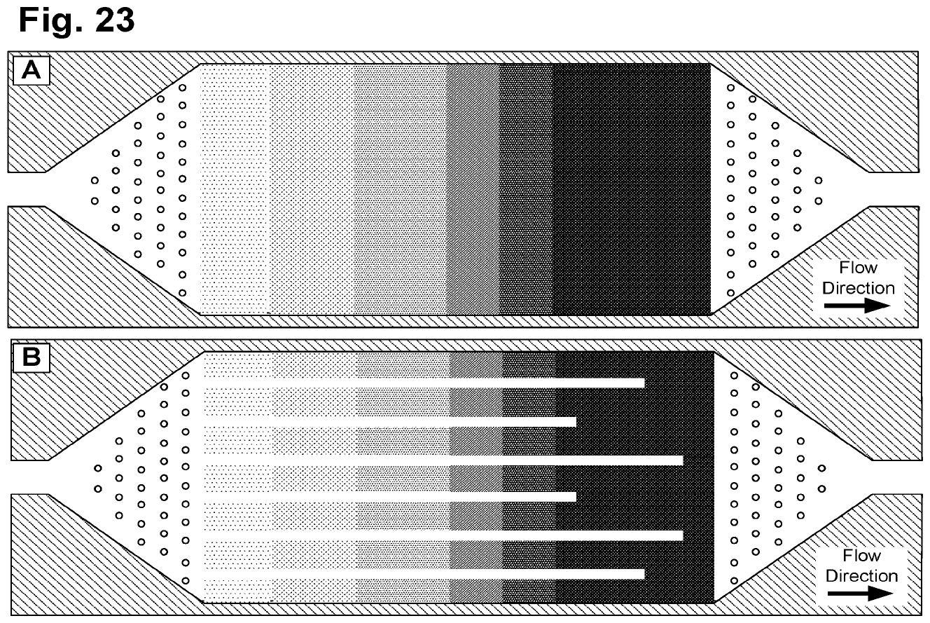

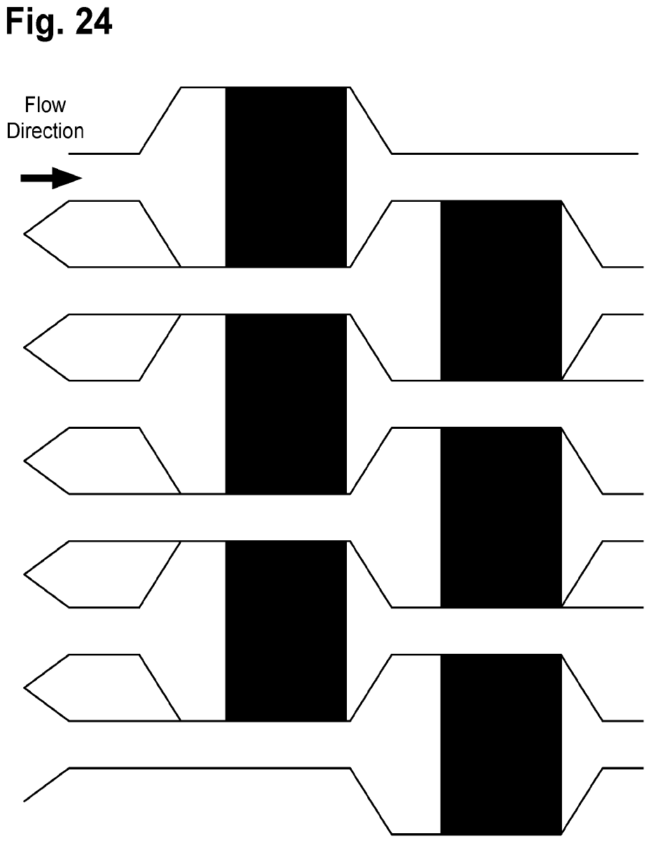

For example, “canals” (as shown in FIG. 23 and FIG. 24) can be generated within the micropillar array by removing sections of pillars in a manner that would allow cells to load further into the array. This would allow the cells to be more spread out over the area of the array rather than all piling up around a localized region. Not only does this alleviate the potential for clogging or flow obstruction within the pillar array regions, but this would also help prevent DNA from bundling into a rope in localized areas with highly densely loaded cells. In turn this facilitates not only the DNA extraction from cells, but also facilitates the removal of the extracted DNA during enzymatic digestion and off-chip collection.

Therefore, building in such canal structures within our micropillar arrays would facilitate cell loading, allow for increased cell capacity within the device, help prevent clogging from localized cell clusters, and improve the on-chip capabilities of DNA extraction.

### Plurality of Channels

In certain aspect, the device or the system of the present disclosure may comprise at least one channel. In some embodiments, the device or the system of the present disclosure may comprise a plurality of channels (e.g., at least 2, 3, 4, 5, 6, 7, 8, 9, 10, 11, 12, 13, 14, 15, 16, 17, 18, 19, 20, or more channels). In some embodiments, such channels may be placed horizontally. In some embodiments, a plurality of channels may be placed in parallel to each other. In other embodiments, a plurality of channels may be placed in a staggered configuration (e.g., FIG. 24).

### Stacked Devices (2 or More Devices Stacked Together, Either Bonded or Unbonded)

Micropillar array regions themselves can be strategically placed in a stacked manner in order to facilitate even better spreading of cellular samples during the steps of cell loading.

For example, the device and system of the present disclosure may include an arrangement of individual or connected/bonded channels or chip cartridges that are vertically stacked on top of one another (in the vertical plane). These stacked chip cartridges or channels can be of identical designs or differing designs, both on a macro scale as well as regarding the microfluidic channels themselves. This could enable a higher processing channel volume/micropillar area without having to expand the horizontal footprint of the channel, and we envision this to be useful for either enhancing or increasing the total yield of gDNA recovered, reducing overall chip cartridge footprint in the horizontal direction, and/or improving throughput of chip cartridge during biological (cellular or nuclei) sample processing,

### Other Variations

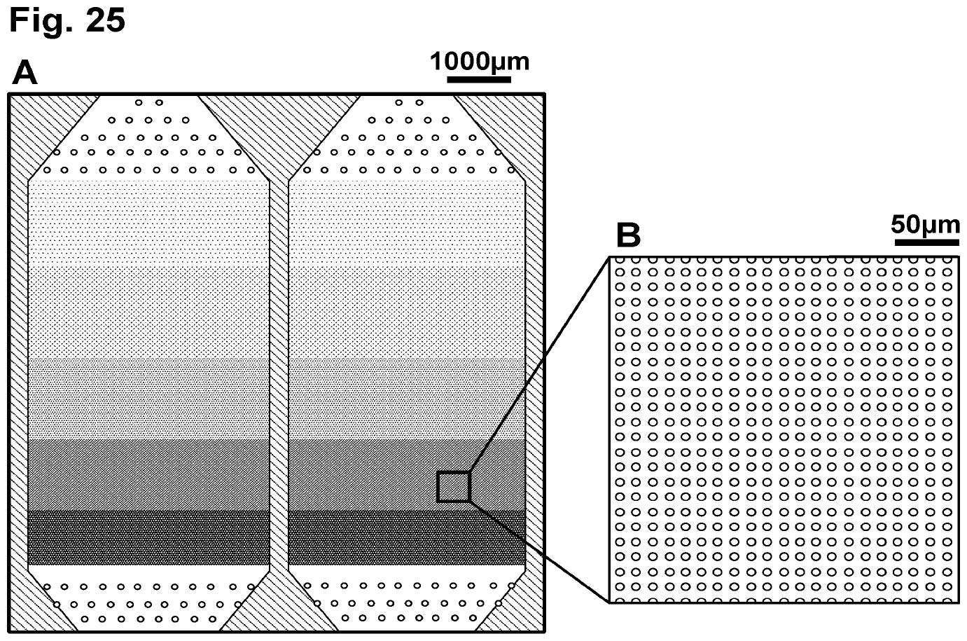

Other variations include those presented in FIG. 25-FIG. 31. Such designs are also described in the Examples section below.

FIG. 29-FIG. 31 comprise a shorter/truncated micropillar array region. In some embodiments, the length (as measured in the direction of the fluid flow) of the shorter/truncated micropillar array region is at least about or less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the length of the channel.

In some embodiments, the length of the shorter/truncated micropillar array region is at least about 1%, 5%, 10%, 15%, 20%, 25%, 30%, 35%, 40%, 45%, or 50% of the length of the channel.

In other embodiments, the length of the shorter/truncated micropillar array region is less than about 1%, 5%, 10%, 15%, 20%, 25%, 30%, 35%, 40%, 45%, or 50% of the length of the channel.

In some embodiments, the length of the shorter/truncated micropillar array region may be in the range of 5 μm to 450 μm. In some embodiments, the length is between 20 μm to 50 μm. In some embodiments, the length is less than 50 millimeters.

In some embodiments, the length of the shorter/truncated micropillar array region may be at least about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, or 450 μm.

### Additional Instruments

The devices, systems, or methods of the present disclosure may further comprise an instrument, an agent, and/or a method that (a) dissociates a tissue into single cells, (b) analyze the cells that enter the microfluidic device, (c) analyze the recovered gDNA or gDNA fragments, or (d) any combination thereof.

In some embodiments, the instrument, agent, and/or method may dissociate a tissue into single cells. Many instruments and agents are available commercially. For example, a tissue can be disrupted into single cells using a sonicator, a homogenizer (including e.g., TissueLyser II (Qiagen), TissueRuptor II (Qiagen), a dounce homogenizer, gentleMACS™ Octo Dissociator with Heaters (Miltenyi Biotec, Cat #130-096-427), gentleMACS™ Dissociator (Miltenyi Biotec, Cat #130-093-235), Singulator 100 (S2 Genomics)), and/or a tissue grinder. In addition, instruments and methods include those described in WO2021/236666A1, WO2018/102471A1, and WO2019/232504A2, each of which is incorporated herein by reference.

In some embodiments, the instrument, agent, and/or method may analyze the cells that enter the microfluidic device. For example, cells or cell nucleic can be sorted by size, morphology, stiffness, cellular mechanical properties, or the presence of a certain cell surface marker. Accordingly, in some embodiments, the devices, systems, or methods of the present disclosure may further comprise a cell fractionator, a flow cytometer, a fluorescence-activated single cell sorting (FACS), a microscope (optical, fluorescent, etc.), or a combination thereof.

In some embodiments, the instrument, agent, and/or method may analyze the recovered gDNA or gDNA fragments. In some embodiments, the recovered gDNA or gDNA fragments may be analyzed by pulse-field gel electrophoresis, hybridization, microarray, amplification, mass spectrometry (e.g., LC-MS), Southern blotting, sequencing, or a combination thereof. Thus, the devices, systems, or methods of the present disclosure may further comprise an instrument that can perform said analysis (e.g., gel electrophoresis, a PCR, a real-time PCR, a mass spectrometer (e.g., LC-MS, LC-MS/MS), a sequencer (e.g., Next Gen Sequencing, Third Gen Sequencing), microarray, or a combination thereof).

### Sequencing

Any of a variety of sequencing reactions known in the art can be used to directly sequence the recovered gDNA fragments. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159). Notably, mass spectrometry (e.g., LC-MS, LC-MS/MS) may be used to sequence DNA (see Chowdhury and Guengerich (2013) Curr Protoc Nucleic Acid Chem 7: Unit-7.1611).

In certain embodiments, detection of gDNA fragments can be accomplished using methods including, but not limited to, sequencing by hybridization (SBH), sequencing by ligation (SBL), quantitative incremental fluorescent nucleotide addition sequencing (QIFNAS), pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al. (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), and the like. High-throughput sequencing methods, e.g., on cyclic array sequencing using platforms such as Roche 454, Illumina Solexa or MiSeq or HiSeq, AB-SOLID, Helicos, Polonator platforms and the like, can also be utilized. High-throughput sequencing methods are described in U.S. Ser. No. 61/162,913, filed Mar. 24, 2009. A variety of light-based sequencing technologies are known in the art (Landegren et al. (1998) Genome Res. 8:769-76; Kwok (2000) Pharmocogenom. 1:95-100; and Shi (2001) Clin. Chem. 47:164-172) (see, for example, U.S. Pat. Publ. Nos. 2013/0274117, 2013/0137587, and 2011/0039304).

Next-generation sequencing (NGS) is a technology for determining the sequence of DNA to study genetic variation associated with diseases or other biological phenomena. Introduced for commercial use in 2005, this method was initially called “massively-parallel sequencing”, because it enabled the sequencing of many DNA strands at the same time, instead of one at a time as with traditional Sanger sequencing by capillary electrophoresis (CE).

Because of the speed, throughput, and accuracy of NGS, NGS enables the interrogation of hundreds to thousands of gDNA fragments at one time in multiple samples, as well as discovery and analysis of different types of genomic features in a single sequencing run, from single nucleotide variants (SNVs), to copy number and structural variants, and even DNA fusions. NGS provides the ideal throughput per run, and studies can be performed quickly and cost-effectively. Additional advantages of NGS include lower sample input requirements, higher accuracy, and ability to detect variants at lower allele frequencies than with Sanger sequencing.

Analyzing the whole genome using next-generation sequencing (NGS) delivers a base-by-base view of all genomic alterations, including single nucleotide variants (SNV), insertions and deletions, copy number changes, and structural variations. Paired-end whole-genome sequencing involves sequencing both ends of a DNA fragment, which increases the likelihood of alignment to the reference and facilitates detection of genomic rearrangements, repetitive sequences, and gene fusions.

In some embodiments, the Illumina “Phased Sequencing” platform, which employs a combination of long and short pair-ends, can be used. In some embodiments, the Illumina “Long Read Assay” that can perform contiguous reads of ˜10 kb-long DNA fragments. In other embodiments, the third-generation single-molecule sequencing technologies (e.g., ONT) can produce much longer reads of DNA sequences.

In some embodiments, the “Deep Sequencing” or high-coverage version of Illumina NGS can be used. Deep Sequencing refers to sequencing a sample multiple times, sometimes hundreds or even thousands of times. The Deep Sequencing allows detection of miRNA, rare clonal types, cells, or microbes comprising as little as 1% of the original sample. Illumina's NovaSeq performs such whole-genome sequencing efficiently and cost-effectively, and its scalable output generates up to 6 Tb and 20 billion reads in dual flow cell mode with simple streamlined automated workflows.

### Microarray

In certain embodiments, analysis of gDNA fragments can be accomplished using microarrays. High-throughput microarrays have been developed to identify and detect the presence of a certain locus or a DNA aberration (e.g., a mutation) (e.g., a substitution, a deletion, an insertion, a duplication, a DNA fusion, a chromosomal fusion, etc.). in a variety of samples, e.g., tissue and cell types.

In some embodiments, covalent attachment of fluorophores can be used to directly label gDNA molecules for use in microarray analyses using e.g., commercially available kits that label DNA.

In certain embodiments, the recovered gDNA in a native form (e.g., chromatin) that comprises a DNA-binding protein (e.g., a transcription factor) can be used in chromatin immunoprecipitation-coupled microarray (also called “ChIP chip”).

### Exemplary Embodiments

1. A microfluidic flow-based device for extracting fragments of genomic DNA in a selected size range from a cell or cell nucleus, comprising
at least one microfluidic channel having at least one inlet port and at least one outlet port to allow a flow in a flow direction from the inlet port toward the outlet port; and
at least one array of micropillars disposed within the microfluidic channel, wherein
   the micropillars have diameters between 3 micrometers and 50 micrometers;
   the micropillars are separated from each other by spacings between 2 micrometers and 150 micrometers;
   the array is bounded within an area between 1 square millimeters and 1600 square centimeters;
   the micropillars have a height between 1-200 micrometers; and
   the micropillars are arranged within the array in a defined configuration.
      2. The device of 1, wherein the micropillars have the shape of circles, ovals, squares, triangles, rectangles, cross shapes, hexagonals, diamonds, polygons, a dome, a pyramid, or any combination of two or more thereof.
      3. The device of 1 or 2, wherein the channel comprises micropillars in:
(a) a random orientation;
(b) a square lattice;
(b) a hexagonal lattice; or
(c) any combination of two or more thereof.
   4. The device of 3, wherein the channel comprises alternating regions of micropillars in a square lattice, micropillars in a random orientation, and micropillars in a hexagonal lattice.
   5. The device of 4, wherein the alternating regions of micropillars in a square lattice and a hexagonal lattice are (a) continuous, or (b) discontinuous such that said regions are separated by a structural region.
   6. The device of any one of 1-5, wherein the micropillars are disposed in a gradient of variable spacings.
   7. The device of 6, wherein the gradient of variable spacings comprises (a) a smooth gradient, (b) a stepped gradient, or (c) a combination of (a) and (b).
   8. The device of any one of 1-7, wherein the channel comprises at least one shorter micropillar array region (e.g., FIG. 29).
   9. The device of 8, wherein the channel comprises at least 2 or at least 3 shorter micropillar array regions, optionally wherein the shorter micropillar array regions are separated by a structural region.
   10. The device of 8 or 9, wherein the length of the shorter micropillar array region is less than about 10%, 15%, 20%, 25%, 30%, 35%, 40%, 45%, or 50% of the length of the channel.
   11. The device of any one of 1-10, wherein the device further comprises one or more canals extending to within said area of the array of micropillars.
   12. The device of 11, wherein the one or more canals are aligned along the flow direction, optionally wherein the one or more canals are straight, branched, curved, or criss-crossed.
   13. The device of 11 or 12, wherein the device comprises at least two canals that have different lengths.
   14. The device of any one of 1-13, wherein said diameters, said spacings, said area, said configuration, or a combination thereof is selected based on type of said cell or cell nucleus.
   15. The device of any one of 1-14, wherein the cell or cell nucleus is of an animal, a fungus, a protist, or a plant, optionally of a human.
   16. The device of any one of 1-15, wherein the cell or cell nucleus is of a leukocyte or a T cell.
   17. The device of any one of 1-16, wherein the device comprises (a) a single-cell sized opening at the apex of the array, and/or (b) a narrower configuration (e.g., micropillar spacing) near the inlet port, which allows extracting fragments of genomic DNA from a single cell.
   18. The device of any one of 1-17, further comprising “gutter” bifurcation channels.
   19. The device of any one of 1-16 and 18, wherein the device comprises (a) a multi-cell sized opening at the apex of the array, and/or (b) a wider configuration (e.g., micropillar spacing) near the inlet port, which allows extracting fragments of genomic DNA from a plurality of cells.
   20. The device of any one of 1-19, further comprising an agent that binds to the cell or cell nucleus.
   21. The device of 20, wherein the agent is an antibody, an antigen-binding fragment thereof, or an aptamer.
   22. The device of 20 or 21, further comprising a secondary channel to the microfluidic channel.
   23. The device of 22, wherein the secondary channel (e.g., FIG. 4) allows lysing the cell and capturing the genomic DNA after the cell is bound to the agent.
   24. The device of any one of 1-23, wherein the micropillars comprise a polymer, plastics, metals, silicons, glass, or any combination of two or more thereof.
   25. The device of any one of 1-24, wherein the micropillars comprise polydimethylsiloxane (PDMS).
   26. The device of any one of 1-25, wherein the micropillars are separated from each other by spacing of in the range of about 2 to 30 micrometers, optionally by spacing of less than or equal to 5 micrometers.
   27. The device of any one of 1-26, wherein the micropillars have the height between 5 to 50 micrometers.
   28. The device of any one of 1-27, wherein the device comprises at least 2 microfluidic channels comprising an array of micropillars.
   29. The device of any one of 1-28, wherein the device comprises at least 6, 12, 8, 12, 24, 48, 96, 384, or 1536 channels.
   30. The device of 28 or 29, wherein
(a) the channels are in a staggered configuration or in a side-by-side configuration in the device; and/or
(b) the channels are in an ordered configuration that aligns with a multi-well plate configuration.
   31. The device of any one of 28-30, wherein the at least 2 microfluidic channels are stacked vertically.
   32. The device of any one of 1-31, further comprising an instrument that isolates a cell and/or a cell nucleus from a tissue.
   33. The device of 32, wherein the instrument comprises a homogenizer, a sonicator, and/or a tissue grinder, optionally wherein the homogenizer is selected from TissueLyser II (Qiagen), TissueRuptor II (Qiagen), a dounce homogenizer, gentleMACS™ Octo Dissociator with Heaters (Miltenyi Biotec, Cat #130-096-427), gentleMACS™ Dissociator (Miltenyi Biotec, Cat #130-093-235), and Singulator 100 (S2 Genomics.
   34. The device of 32 or 33, wherein the tissue is fresh, frozen, flash-frozen, formalin-fixed paraffin-embedded (FFPE), or preserved in optimal cutting temperature compound (OCT).
   35. The device of any one of 1-34, further comprising an instrument that sorts a cell or a cell nucleus.
   36. The device of 35, wherein the instrument comprises a flow cytometer, a fluorescence-activated cell sorting (FACS) instrument, and/or a microscope.
   37. The device of any one of 1-36, further comprising at least one agent that facilitates lysis of a cell or a cell nucleus (e.g., a lysis buffer a denaturant, and/or a wash buffer).
   38. The device of any one of 1-37, further comprising a digestion buffer, one or more enzymes, a DNA labeling agent (e.g., biotin, a fluorescent molecule, a barcode), a hybridization agent (e.g., a primer (e.g., labeled with a detectable marker, e.g., fluorescent molecule, a biotin), and/or a wash buffer.
   39. The device of 38, wherein the one or more enzymes are selected from a DNA-cleaving enzyme, a DNA-modifying enzyme (e.g., DNA methyltransferase, terminal transferase, T4 DNA ligase, DNA polymerase), and an enzyme that modifies DNA-associated proteins.
   40. The device of 38 or 39, wherein the one or more enzymes are selected from a restriction enzyme, an endonuclease, a homing endonuclease, a transposase, type-II CRISPR-Cas9 protein, and dsDNA Fragmentase.
   41. The device of any one of 38-40, wherein the one or more enzymes comprise one or more restriction enzymes.
   42. The device of any one of 1-41, further comprising at least one instrument that facilitates DNA cleavage and/or DNA release from the channel.
   43. The device of 42, wherein the at least one instrument is selected from a sonicator, a nebulizer, a light source (which emits e.g., visible light, UV light, X-rays, gamma rays, or ionizing radiation), or any combination of two or more thereof.
   44. The device of any one of 1-43, wherein the micropillars are collapsible, meltable, mechanically retractable, dissolvable, mechanically peeled off, mechanically bendable, and/or electrically bendable.
   45. The device of any one of 1-44, further comprising at least one apparatus that analyzes the isolated DNA.
   46. The device of 45, wherein the at least one apparatus is selected from a microscope, a fluorescence microscope, an optical microscope, a microarray, a mass spectrometer (e.g., LC-MS, LC-MS/MS), a PCR, a real-time PCR, a gel electrophoresis, a capillary gel electrophoresis, and a sequencer (e.g., NGS, Third Gen sequencer).
   47. The device of any one of 1-46, further comprising a robotic sample preparation platforms and/or a plate reader.
   48. The device of any one of 1-47, wherein the recovered genomic DNA comprises at least one DNA binding protein.
   49. The device of 48, wherein the at least one DNA binding protein comprises a histone, a transcription factor, a replication protein, a DNA repair protein, a DNA modifying enzyme (e.g., a DNA methyltransferase), and/or a nuclease.
   50. A system comprising at least one device of any one of 1-49, further comprising at least one fluid control module adapted to connect to at least one inlet port.
   51. The system of 50, wherein the fluid control module comprises a pipette or a syringe.
   52. The system of 50, wherein the fluid control module comprises a pressurized air source (e.g., compressed air), controlled pressure source, controlled pneumatic pressure source, pressure driven pump, syringe pump, vacuum pump, or a peristaltic pump.
   53. The system of any one of 50-52, further comprising one or more collection reservoirs adapted to connect to the outlet port, optionally wherein the one or more collection reservoirs comprise a sample collection reservoir and a waste collection reservoir.
   54. The system of any one of 50-53, further comprising a controlled voltage source for electrophoretically driving DNA within the microfluidic channel.
   55. The system of any one of 50-54, further comprising at least one temperature control module, optionally wherein (a) the at least one temperature control module can heat different portions of the device at different temperatures, and/or (b) the at least one temperature control module can be controlled in a time-controlled manner (e.g., can heat for a certain duration of time).
   56 The system of 55, wherein the at least one temperature control module can set the temperature of the device in the range of about 2° C.-100° C.
   57. The system of any one of 50-56, wherein the system comprises at least 2 devices, optionally wherein the at least 2 devices are stacked vertically.
   58. A method of selecting parameters for extracting fragments of genomic DNA having a desired size metric from a cell or cell nucleus, comprising
processing a sample comprising at least one cell or cell nucleus through the device of any one of 1-49 or the system of any one of 50-57, at least once using parameters that comprise
   a concentration for each of said one or more enzymes;
   a number of said one or more enzymes;
   a buffer composition for said digestion buffer;
   a digestion time; and
   a digestion temperature;
determining a fragment size metric for a collected sample; and, based on said fragment size metric,
   if said fragment size metric corresponds to the desired size metric, selecting the used parameters;
   if said fragment size metric is lesser than the desired size metric, reducing said concentration, reducing said number, reducing said digestion time, changing said digestion temperature to reduce enzyme efficiency, changing said buffer composition to reduce enzyme efficiency, or a combination thereof until said fragment size metric is not lesser than the desired size metric; and
   if said fragment size metric is greater than the desired size metric, increasing said concentration, increasing said number, increasing said digestion time, changing said digestion temperature to increase enzyme efficiency, changing said buffer composition to increase enzyme efficiency, or a combination thereof until said fragment size metric is not greater than the desired size metric.
      59. The method of 58, wherein said fragment size metric is fragment size mean or fragment size median.
      60. The method of 58 or 59, wherein said processing comprises loading the sample into the device and washing at least one cell or cell nucleus in the device using the wash buffer.
      61. The method of any one of 58-60, further comprising inactivating the one or more enzymes.
      62. The method of 61, wherein said inactivation comprises heat inactivation.
      63. A method of isolating a cell or a cell nucleus from a sample, the method comprising
flowing the sample through the device of any one of 1-49, or the system of any one of 50-57; and
collecting the cell or cell nucleus entrapped in the device.
   64. The method of 63, further comprising lysing the cell membrane or cell wall but leaving the nuclear membrane intact.
   65. The method of 63 or 64, further comprising washing the entrapped cell or the cell nucleus.
   66. The method of any one of 63-65, wherein the cell or the cell nucleus is of an animal, a fungus, a protist, or a plant, optionally of a plant.
   67. The method of any one of 63-66, wherein the sample is selected from blood, plasma, lymph, saliva, urine, in vitro cell culture, and tissue homogenates, optionally wherein the tissue homogenate is a plant tissue homogenate.
   68. The method of any one of 63-67, wherein the sample further comprises a blocking agent (e.g. BSA, ionic detergent, another charged agent, PEG, or PEO polymer.
   69. A method of isolating a genomic DNA of a large size (e.g., intact gDNA, whole chromosome, or a fragment of gDNA greater than 500 kb), the method comprising processing a sample comprising at least one cell or cell nucleus through the device of any one of 1-49 or the system of any one of 50-57, optionally wherein the device comprises micropillars that are collapsible, meltable, mechanically retractable, dissolvable, mechanically peeled off, mechanically bendable, and/or electrically bendable.
   70. A method of isolating fragments of genomic DNA having a selected size metric from at least one cell or cell nucleus, comprising:
(a) processing a sample comprising at least one cell or cell nucleus through the device of any one of 1-49, or the system of any one of 50-57; and
(b) collecting the fragments of genomic DNA isolated via said processing.
   71. The method of 70, wherein the fragments of gDNA are produced via digestion with a DNA-cleaving enzyme, sonication, nebulization, altering flow conditions, light method, mechanical method, or a combination of two or more thereof.
   72. The method of 70 or 71, wherein the method comprises using the parameters selected via the method of any one of 58-62.
   73. The method of any one of 70-72, further comprising the method of isolating a cell or a cell nucleus of any one of 63-68.
   74. The method of any one of 70-73, further comprising
(a) washing the entrapped cell or the cell nucleus prior to the lysis;
(b) washing the genomic DNA prior to digestion; and/or
(c) inactivating the one or more enzymes (e.g., heat inactivation).
   75. The method of any one of 70-74, wherein the fragments of genomic DNA are in the range of about 1 kb to 500 kb.
   76. The method of any one of claims 70-75, wherein the fragments of genomic DNA are in the range of 200 kb, 30 kb, 25 kb, 14 kb, 10 kb, or 8 kb.
   77. The method of any one of 70-76, wherein the genomic DNA is from a single cell or a single cell nucleus.
   78. The method of any one of 70-77, wherein collecting the fragments of genomic DNA comprises removing the fragments from the collection reservoir via manual pipetting or through tubing.
   79. The method of any one of 70-78, wherein collecting the fragments of genomic DNA comprises removing the fragments from the collection reservoir via an electrophoretic channel connected to the collection reservoir.
   80. The method of any one of 70-79, further comprising sequencing, amplifying, hybridizing, end-labeling, and/or barcoding the genomic DNA.
   81. The method of any one of 70-80, wherein the cell or a cell nucleus is of a an animal, a fungus, a protist, or a plant, optionally a human.
   82. The method of any one of 70-81, wherein the cell or a cell nucleus is of a leukocyte or a T cell.
   83. The method of any one of 70-82, wherein the sample comprises blood, plasma, lymph, saliva, urine, semen, in vitro cell culture, and/or a tissue homogenate.
   84. The method of any one of 70-83, wherein the sample comprises partially or fully purified cell or cell nucleus.
   85. A method of detecting a mutation in a genome, the method comprising:
(a) isolating a genomic DNA or fragments thereof according to the method of any one of 69-84; and
(b) analyzing the recovered DNA, thereby detecting the mutation.
   86. The method of 85, wherein the mutation comprises a substitution, a deletion, an insertion, a duplication, a DNA fusion, and/or a chromosomal fusion.
   87. The method of 85 or 86, wherein the DNA is analyzed using one or more selected from: gel electrophoresis, capillary electrophoresis, hybridization, PCR, a real-time PCR, a mass spectrometry (e.g., LC-MS, LC-MS/MS), Southern blotting, sequencing (e.g., NGS, Third Gen Sequencing), a microarray, and a combination of any two or more thereof.
   88. The method of any one of 85-87, wherein the genomic DNA is of a an animal, a fungus, a protist, or a plant, optionally a human.
   89. The method of any one of 69-88, wherein the sample or the buffer (e.g., lysis buffer, wash buffer, elution buffer, digestion buffer, etc.) comprises a blocking agent (e.g. BSA, ionic detergent, another charged agent, PEG, or PEO polymer).
   90. The device of any one of 1-49 or the system of any one of 50-57, wherein the at least one channel comprises a blocking agent (e.g. BSA, ionic detergent, another charged agent, PEG, or PEO polymer), optionally wherein the blocking agent coats the inner surface of the channel and/or the micropillars within the channel.

### EXAMPLES

The disclosure will be further illustrated with reference to the following specific examples. These examples are given by way of illustration and are not meant to limit the disclosure or the claims that follow.

### Example 1: Fabrication of a Microfluidic Device

To fabricate the master molds, Microposit S1813 photoresist (Shipley; Marlborough, MA) is spun on silicon on insulator (SOI) wafers (Ultrasil; Hayward, CA) and exposed by UV contact lithography (EVG620, EVG Group; Albany, NY). The exposed resist is developed in 726MIF developer (Microchemicals) and the pattern is transferred into the 20 μm-thick top silicon layer by Bosch process in a Unaxis SLR 770 deep reactive ion etching system (Unaxis USA Inc.; St. Petersburg, FL). A monolayer of (1H, 1H, 2H, 2H Perfluorooctyl) Trichlorosilane is deposited on the etched wafers in a MVD100 molecular wafer deposition system (Applied Microstructures; San Jose, CA) to prevent sticking of the PDMS to the mold. Sylgard 184 (Dow Corning; Midland, MI) PDMS base resin is mixed with the curing agent at a 10:1 ratio, degassed under vacuum at room temperature, poured onto the master, and cured for 45 min at 150° C. The elastomer casting is then peeled off the mold and access holes to the input and outputs of the microchannels are created with a 1.5 mm biopsy punch (Sklar Instruments; West Chester, PA). To complete channel fabrication, the patterned PDMS is treated with oxygen plasma for 1 min and bonded to a 170-μm thick fused silica wafer (Mark Optics; Santa Ana, CA).

### Example 2: Example Protocol for Extracting DNA at 10 kb

1. Preparation of cell samples. Any sample must be in a solution in order to be effectively loaded onto the device. Preparation of two sample types are detailed below.
Blood: Using a lancet, a human finger is pricked to generate 1-2 drops of blood (˜100 uL). The drops of blood are then transferred into a tube with 500 μL of 1×PBS containing 5 mM EDTA and gently mixed. The cell solution is then stored on ice until loading onto the device.
Animal cells (e.g., mammalian cells): Immortalized or primary animal cells (e.g., mammalian cells) growing in culture can be prepared using standard methods. Briefly, media is aspirated from the plate they are growing on, and the cells are washed once with 1×PBS. The cells are then treated with trypsin+EDTA to release them from the plate. The cells are then washed with media, and spun down for 2 minutes at 2,000 RPM. The supernatant is removed, and the cells are resuspended in media. The resuspended cells are then filtered using a 40 micrometers cell strainer and stored on ice until ready to load onto the device.
Plant cells: Removal of plant compounds or secondary metabolites from a crude nuclei plant preparation.

When sequencing plant DNA, nuclei must first be prepared from the plant tissue. A method to release plant nuclei is often used that crudely releases the nuclei from the cell walls but also contains unwanted plant compounds including secondary metabolites such as starch and polyphenols. These particles are difficult to remove and are unwanted as they inhibit sequencing reactions.

The crude samples are loaded into the microfluidic device that has micropillars that will catch the nuclei but allow for the much smaller plant compounds or secondary metabolites such as starch or polyphenols to escape the array. Due to the small particle size of these compounds, a final micropillars spacing size of ˜ 5 micrometers is used for effective separation. The plant nuclei are thus isolated/entrapped within the array and purified from the unwanted plant compounds.

2. Preparation of microfluidic device. The microfluidic device (made of PDMS) is first primed prior to cell loading. The device is plasma cleaned briefly for 1-3 minutes, and then back loaded with 100% ethanol to fill the all the channels. Next, the input port is connected with the pressure and solution delivery mechanism. In this case, an apparatus is plugged directly into the device, which contains space to hold a volume of solution and caps with a hose connected to a pressure value, regulator, and tank.

3. Loading of the cells. Optimal loading is achieved when the micropillar array is filled with enough cells to produce the desired gDNA yield without overloading. Cells are loaded at psi of 0.1-0.5 for seconds to minutes depending on the concentration of the cells. After loading is complete, the solution is exchanged from the apparatus with 1×PBS via pipetting and allowed to flow at 0.1-0.5 psi for a few minutes. At this point, only cells are captured in the device—all other components of the media or any other particle not caught in the array has been washed out to the output port.

4. Lysis of the cells. To lyse the cells, the apparatus solution is exchanged with a suitable lysis buffer—in this case 1% SDS in 1×PBS (any other chemical lysis buffer would work) for 5 minutes under a pressure of 0.1-0.5 psi. Lysis is immediate but is allowed to continue for several minutes to ensure complete lysis. Next, the buffer in the apparatus is washed and exchanged with 1×PBS, and then the device is washed with this solution for 5 minutes at 0.1-0.5 psi.

5. Fragmentation and release of the gDNA. To release the gDNA trapped on the micropillars at fragment lengths of 10 kb, the following buffer is used: 10 mM Tris-HCl, 50 mM NaCl, 10 mM MgCl2 with 1 mM DTT. To 200 μL of this buffer, the following enzymes are added: 0.2 U EcoRV, 0.2 U EcoRI, 0.2 U HindIII and 0.2 U ScaI. The buffer is exchanged in the apparatus and allowed to flow for 15 minutes at 0.3-0.4 psi at 27° C. After 15 minutes, the pressure is increased to 1.6 psi for 5-10 minutes until all the solution has run through the device. The gDNA is then collected off the chip and heated for 15′ at 80° C. to inactivate the enzymes, then cooled on ice and stored a 4° C.

6. Analysis of gDNA. The sample is first quantified using a Qubit to determine yield and concentration. If needed, the sample is then diluted to 400 ng/uL for fragment size analysis using a Femto Pulse pulse-field capillary electrophoresis system to confirm quality and size of fragmentation.

Specific examples demonstrating how varying the abovementioned variables in different combinations can produce various gDNA fragment sizes are outlined below.

All data below were generating using culture HeLa cells and a device with the following parameters and dimensions:
1 input to 4 outputs for each device
4 mm Wide×3.6 mm Long array region for each channel in each device
Micropillar-micropillar (center to center) spacing within array: continuous gradient from 33.3 to 18 micrometers
Micropillar diameter: 6.3 micrometers
Micropillar height: 18-20 micrometers

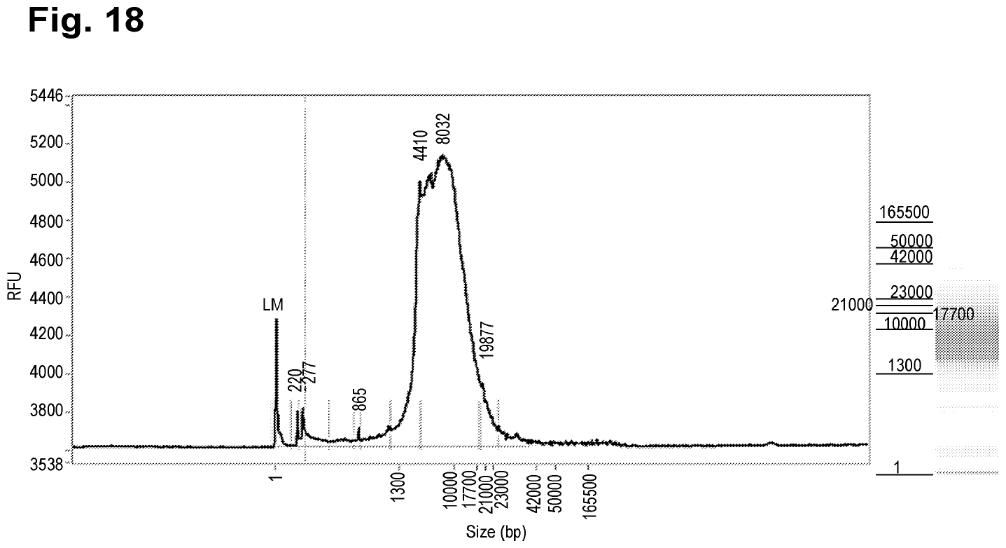

### Example 3 (FIG. 18): Tuning Fragment Size with Type II Restriction Enzymes: 8 kb Range

To recover gDNA with a main peak around 8 kb, a reaction mixture was prepared containing the following:
4 type II restriction endonucleases (0.8-1.2 U/uL final)
in a buffer containing
   Tris-HCl (10 mM)
   NaCl (50 mM)
   MgCl2 (10 mM)
   DTT (1 mM)

The buffer was introduced to the microfluidic channel through the apparatus by pipetting or a syringe pump. The device was set to a controlled reaction temperature (27 C.°). The reaction was allowed to proceed under a flow pressure of 0.3 psi for 15-20 minutes, and then 5-15 minutes at 1.3-1.8 psi.

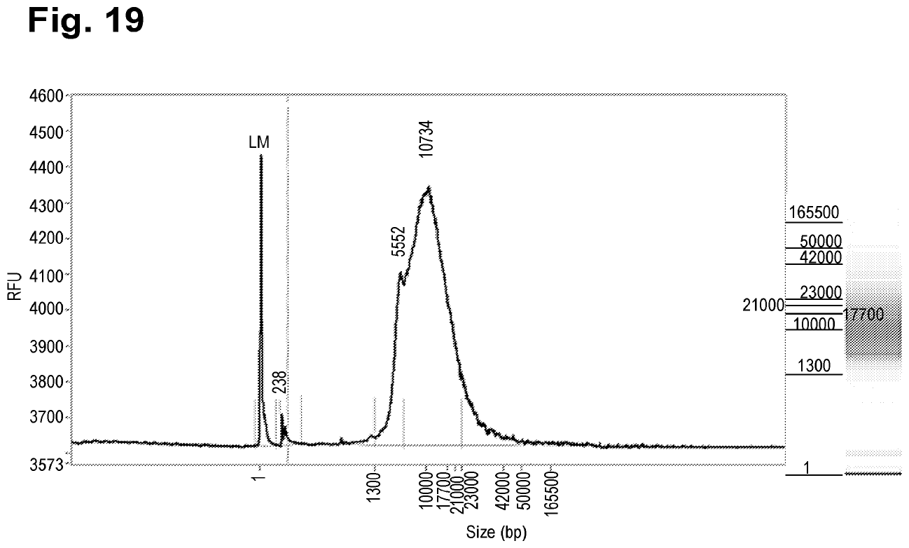

### Example 4 (FIG. 19): Tuning Fragment Size with Type II Restriction Enzymes: 10 kb Range

To recover gDNA with a main peak around 10 kb, a reaction mixture was prepared containing the following:
4 type II restriction endonucleases (0.7-0.9 U/uL final)
in a buffer containing
   Tris-HCl (10 mM)
   NaCl (50 mM)
   MgCl2 (10 mM)
   DTT (1 mM)

The buffer was introduced to the microfluidic channel through the apparatus by pipetting or a syringe pump. The device was set to a controlled reaction temperature (27 C.°). The reaction was allowed to proceed under a flow pressure of 0.3 psi for 15-20 minutes, and then 5-15 minutes at 1.3-1.8 psi.

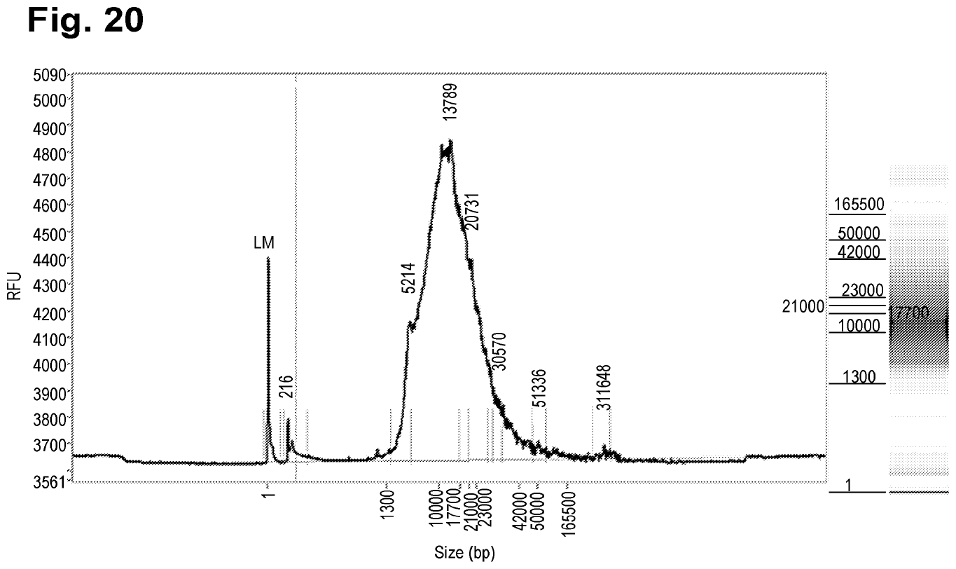

### Example 5 (FIG. 20): Tuning Fragment Size with Type II Restriction Enzymes: 14 kb Range

To recover gDNA with a main peak around 14 kb, a reaction mixture was prepared containing the following:
4 type II restriction endonucleases (0.6-0.7 U/uL final)
in a buffer containing
   Tris-HCl (10 mM)
   NaCl (50 mM)
   MgCl2 (10 mM)
   DTT (1 mM)

The buffer was introduced to the microfluidic channel through the apparatus by pipetting or a syringe pump. The device was set to a controlled reaction temperature (27 C.°). The reaction was allowed to proceed under a flow pressure of 0.3 psi for 15-20 minutes, and then 5-15 minutes at 1.3-1.8 psi.

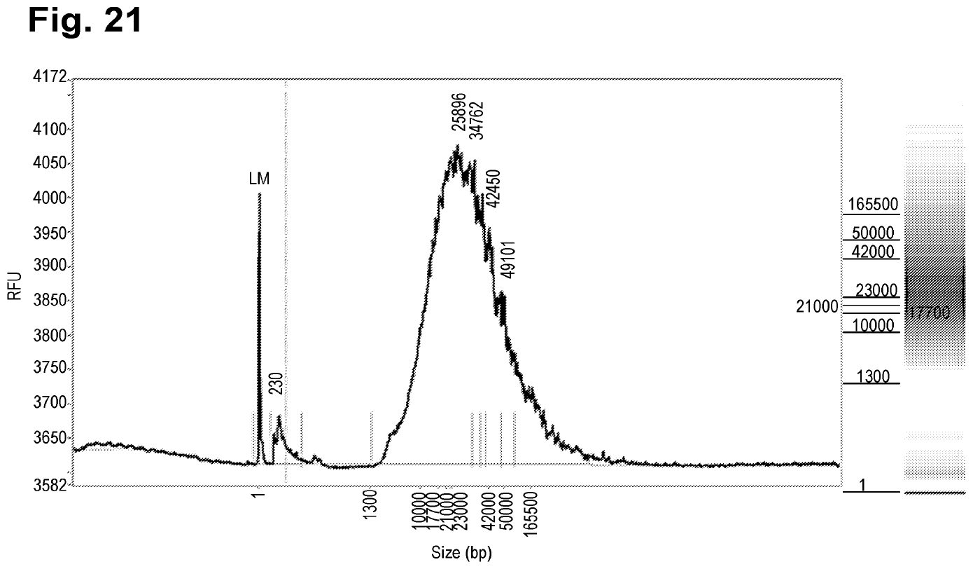

### Example 6 (FIG. 21): Tuning Fragment Size with Type II Restriction Enzymes: 30 kb Range

To recover gDNA fragments ranging from 1-165 kb with a main peak around 25-30 kb, a reaction mixture was prepared containing the following:
one type II restriction enzyme (0.8 U/uL final)
in a buffer containing
   Tris-HCl (10 mM)
   NaCl (50 mM)
   MgCl2 (10 mM)
   DTT (1 mM)

The buffer was introduced to the microfluidic channel through the apparatus by pipetting or a syringe pump. The device was set to a controlled reaction temperature (27 C.°). The reaction was allowed to proceed under a flow pressure of 0.35-0.4 psi for 15 minutes, then 5 minutes at 1.6 psi.

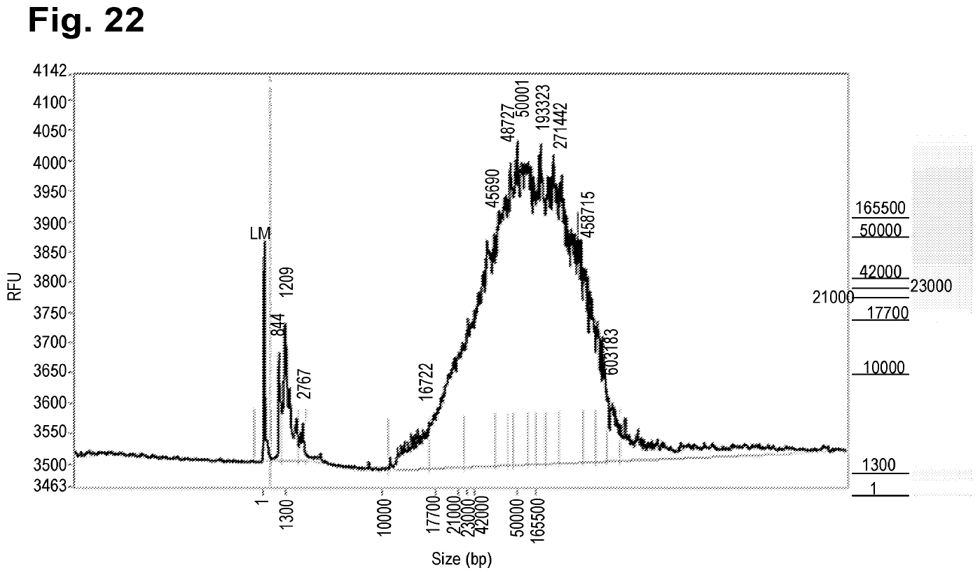

### Example 7 (FIG. 22): Tuning Fragment Size with Type II Restriction Enzymes: 200 kb Range

To recover gDNA fragments with a main peak around 200 kb, a reaction mixture was prepared containing the following:
one type II restriction enzymes (0.2 U/uL final)
in a buffer containing
   Tris-HCl (10 mM)
   NaCl (50 mM)
   MgCl2 (10 mM)
   DTT (1 mM)

The buffer was introduced to the microfluidic channel through the apparatus by pipetting or a syringe pump. The device was set to a controlled reaction temperature (27 C.°). The reaction was allowed to proceed under a flow pressure 0.2 psi for 15 minutes, then 0.3 psi for 5 minutes, then 1.6 psi for 15 minutes.

Many more possible fragment sizes, ranging from 100s of base pairs to Mbs can be tuned by adjusting the aforementioned parameters.

### Example 8: Analysis of the Recovered DNA (Real-Time PCR)

A representative protocol involves the following: the recovered gDNA is diluted 1:20 and 2 μl used in the realtime PCR with the forward and reverse PCR primers that hybridizes to a certain locus. The gDNA fragment is added to a master mix containing 10 ul of 2× PowerSYBR green master mix (Applied Biosystem), 1 ul of 10 uM primers mix, and 7 ul of water. The reaction is incubated in an Applied Biosystems 7500 realtime PCR system at 95° C. for 10 min, followed by 45 cycles of 95° C. for 15 s, 60° C. for 15 s and 72° C. for 32 s. After that, dissociation stage/melting curve analysis is performed.

When analyzed, the gDNA fragments recovered using the devices, systems, and/or methods of the present disclosure surprisingly outperformed the traditionally prepared (e.g., CsCl-purified) gDNA fragments in the same real-time PCR reaction.

### Example 9: gDNA Amplification

We have demonstrated successful on-chip and off-chip DNA amplification.

For On-Chip, we have demonstrated isothermal amplification (See Tian et al. (2018) PLOS ONE 13 (2): e0191520, which is incorporated herein by reference) using Multiple Displacement Amplification (MDA). We then successfully collected the amplified DNA and performed whole exome sequencing on Illumina sequencers as a demonstration of coverage breadth of the method, as well as showing reduction of amplification bias compared to MDA in-tube (see also Example 10).

For Off-Chip, we have successfully collected purified DNA from the chip device post extraction and DNA fragmentation/cleavage. We have taken the DNA sample and then demonstrated the ability to PCR and qPCR the DNA and then successfully sequence the sample on Illumina sequencers.

A combination of both on-chip amplification (either PCR or isothermal) and off-chip amplification (either PCR or isothermal) is also possible.

### Example 10: Analysis of the Recovered DNA (Next-Generation Sequencing)

The recovered gDNA fragments were sequenced using the Next-Generation Sequencing (NGS) according to the manufacturer's instructions (Illumina, San Diego, CA). The results were surprisingly and unexpectedly superior to the sequencing performed using traditionally prepared (e.g., CsCl-purified) gDNA fragments.

### Example 11: Exemplary Devices

Exemplary devices that have been made are shown in the drawings presented herein (e.g., FIG. 3, FIG. 5, FIG. 23 (A and B), FIG. 24, and FIG. 25-FIG. 31). Provided below are additional descriptions for select designs.

One aspect/goal of various designs is to improve sample loading (sample comprising at least one cell or cell nucleus). The improved sample loading includes the ability to better spread out where cells or nuclei loaded into the channel will become arrested/entrapped within the micropillar array in a manner sufficiently far enough from one another as to mitigate/minimize the effects of clogging the fluid flow. The following devices are designed for better sample loading.

While any lattice of micropillars (including a random lattice) will work in extracting gDNA or fragments thereof, the square and hexagonal lattices provide a unique opportunity for better sample loading.

Regarding the gradients (both smooth and stepped gradients) described below, various numbers of gradients have been tested. In particular, the stepped gradients with as little as 2 steps (2 total but adjacent micropillar array regions, each with different set spacing), and as much as 30 steps in a gradient have been tested.

FIG. 25 shows a device having at least one channel with a square lattice of micropillars, where the pillars were placed in a way to have clear rows and columns at specified distances apart from each other. The square lattice refers to a lattice of micropillars in which each gap (between pillars) is lined up with the next adjacent gap, for regions of the micropillar array that is meant to be at a uniform spacing. In other words, each pillar is lined up to be in one line of vision to the next pillar. This device was installed with a smooth gradient of varying spacings of the pillars in square lattice formation. This device was also installed with a smaller range of pillar spacing as a gradient. The gradient with a decrease in micropillar spacing near the outlet port may be desired if the intended genomic DNA is of higher molecular weight because such a gradient allows better spreading of cells within the sample as well as cells away from other smaller particles in the sample.

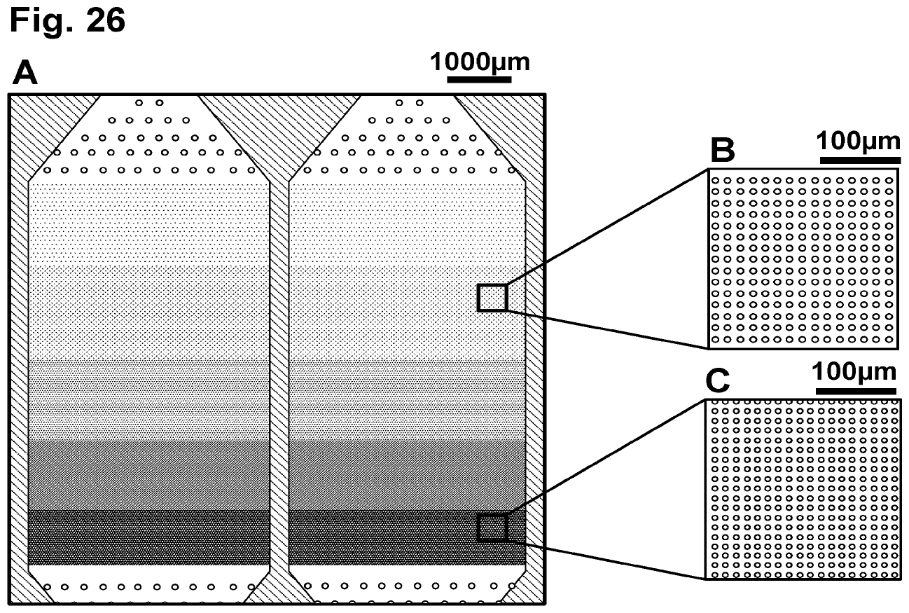

FIG. 26 shows a device having a square lattice of micropillar array that contained a stepped gradient in spacings of micropillars. Here, the spacings were held uniform over a periodicity before they were changed to the next periodicity, rather than having one smooth transition of spacings from the top of the micropillar array to the bottom.

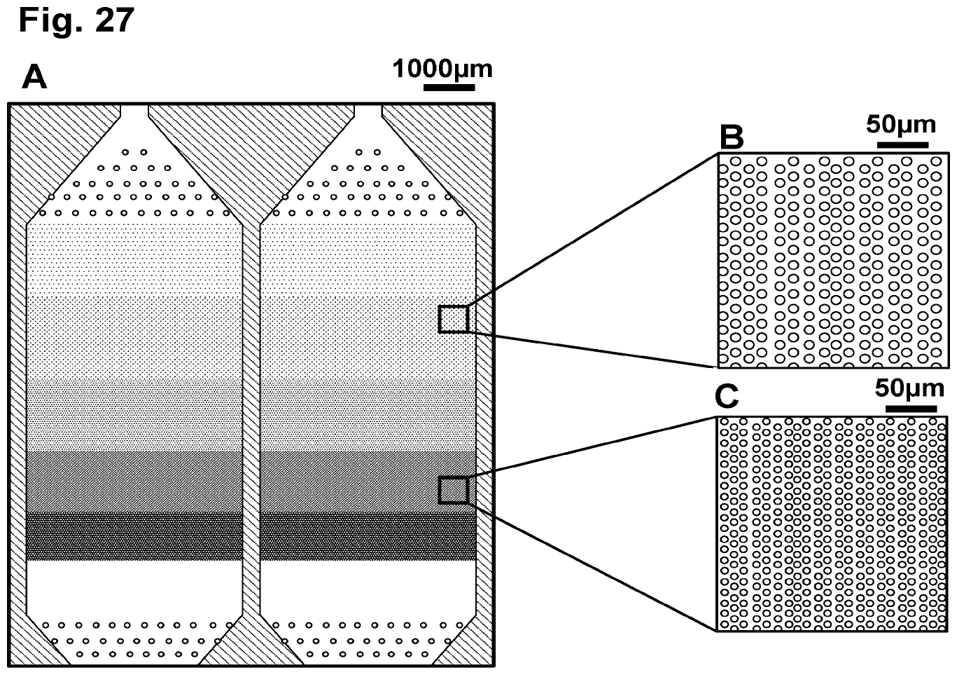

FIG. 27 shows a device having a hexagonal lattice micropillar array. The hexagonal lattice refers to a lattice of micropillars in which each gap is lined up to be intersecting a pillar. In other words, pillars and gaps are staggered to maximize the likelihood or probability that any particle flowing through the micropillar array will encounter an object (e.g., the next micropillar that is adjacent and/or downstream). The gradient of pillar spacings can be either smooth or stepped.

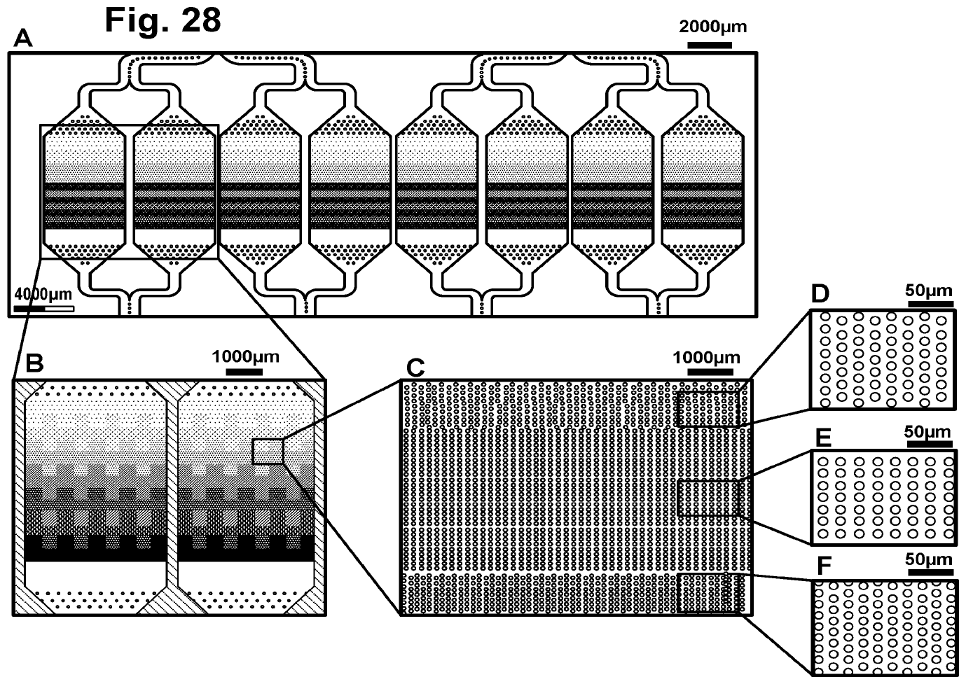

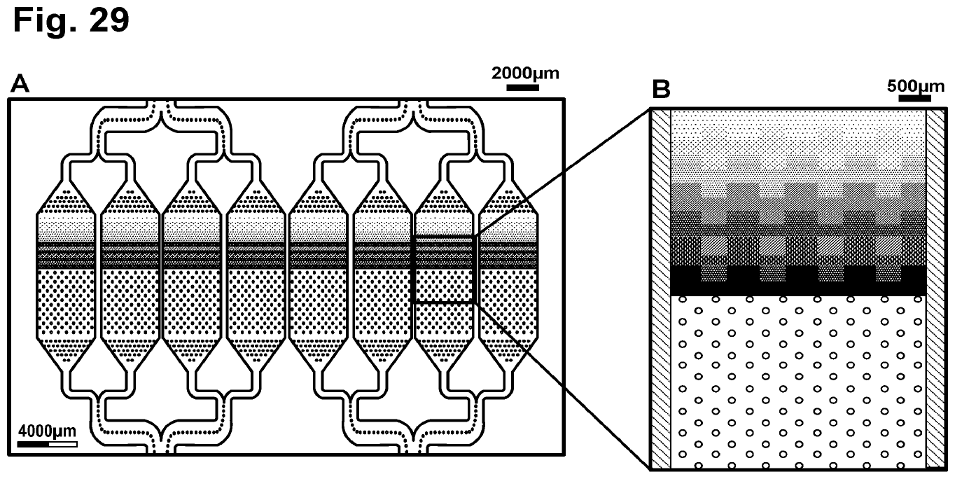

FIG. 28 shows a device that was installed with interweaving square-lattice and hexagonal-lattice regions.

FIG. 29 shows a device that was installed with a shorter array of micropillars as compared with the devices of FIG. 25-FIG. 28. The shorter length of the micropillar array may improve DNA yield/quantity of DNA collected from the microfluidic chip cartridges. the area within the channel that is not occupied by the shorter array of micropillars may comprise certain structures to prevent collapsing of the chip cartridge, which may be necessary for a device made with a soft material such as PDMS. However, if the structural integrity is not a concern for a particular device (e.g., made with plastics or metals), such area may be empty (i.e., devoid of structures or micropillars). As used herein, the area not occupied by the shorter array of micropillars is referred to as a “structural region” even if the area is empty and devoid of any structures.

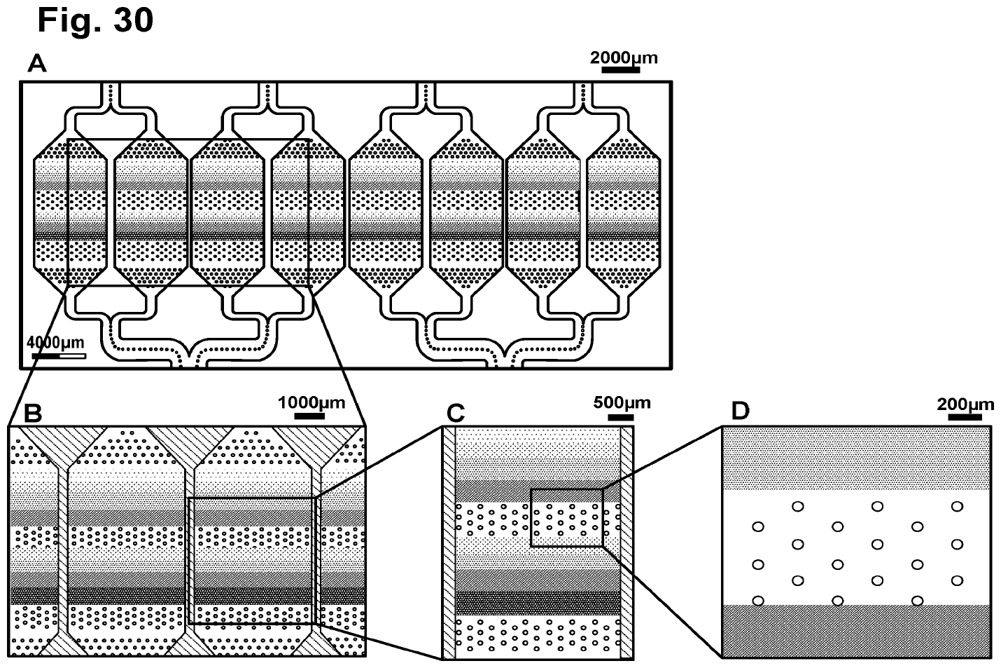

FIG. 30 shows a device installed with weaving micropillar regions with non-micropillar/empty/support structure regions. Here, two micropillar array regions were separated by a distance in support structures. Having detached two micropillar arrays may improve sample loading by further spacing smaller cells or nuclei away from one another and thereby mitigate/minimize fluid flow constrictions from heavily clumped/roped DNA during the sample lysis and DNA extraction steps.

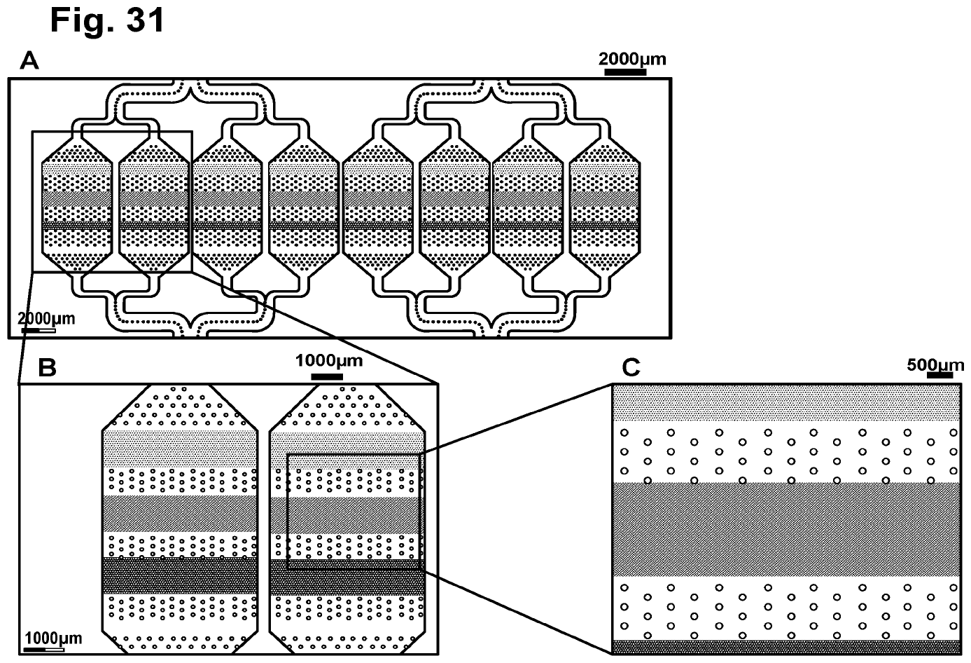

FIG. 31 shows 3 micropillar array regions that are separated by support structures. 3 weaving micropillar array regions may perform better than 2 weaving micropillar array regions of FIG. 30. In addition, more repeat micropillar array regions may lead to better outcomes for the ultimate DNA yield collected from the chip cartridges after DNA cleaving.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

### INCORPORATION BY REFERENCE

All U.S. patents, and U.S. and PCT patent application publications mentioned herein are hereby incorporated by reference in their entirety as if each individual patent or patent application publication was specifically and individually indicated to be incorporated by reference. In case of conflict, the present application, including any definitions herein, will control.

### EQUIVALENTS

Those skilled in the art will recognize or be able to ascertain using no more than routine experimentation many equivalents to the specific embodiments of the present invention described herein. Such equivalents are intended to be encompassed by the following claims.

1-90. (canceled)

91. A microfluidic flow-based device for extracting fragments of genomic DNA in a selected size range from a cell or cell nucleus, comprising
at least one microfluidic channel having at least one inlet port and at least one outlet port to allow a flow in a flow direction from the inlet port toward the outlet port; and
at least one array of micropillars disposed within the microfluidic channel, wherein the micropillars have diameters between 3 micrometers and 50 micrometers;
the micropillars are separated from each other by spacings between 2 micrometers and 150 micrometers;
the array is bounded within an area between 1 square millimeters and 1600 square centimeters;
the micropillars have a height between 1-200 micrometers; and
the micropillars are arranged within the array in a defined configuration.

92. The device of claim 91, wherein the micropillars have the shape of circles, ovals, squares, triangles, rectangles, cross shapes, hexagonals, diamonds, polygons, a dome, a pyramid, or any combination of two or more thereof.

93. The device of claim 91, wherein the channel comprises micropillars in:
(a) a random orientation;
(b) a square lattice;
(c) a hexagonal lattice; or
(d) any combination of two or more thereof.

94. The device of claim 91, wherein the micropillars are disposed in a gradient of variable spacings, wherein the gradient of variable spacings comprises (a) a smooth gradient, (b) a stepped gradient, or (c) a combination of (a) and (b).

95. The device of claim 91, wherein the micropillars are separated from each other by spacing of in the range of about 2 to 30 micrometers, optionally by spacing of less than or equal to 5 micrometers.

96. The device of claim 91, wherein the device comprises at least 6, 12, 8, 12, 24, 48, 96, 384, or 1536 channels, and optionally wherein:
(a) the channels are in a staggered configuration or in a side-by-side configuration in the device; and/or
(b) the channels are in an ordered configuration that aligns with a multi-well plate configuration.

97. The device of claim 91, further comprising a digestion buffer, one or more enzymes, a DNA labeling agent (e.g., biotin, a fluorescent molecule, a barcode), a hybridization agent (e.g., a primer (e.g., labeled with a detectable marker, e.g., fluorescent molecule, a biotin), and/or a wash buffer, optionally wherein the one or more enzymes are selected from a DNA-cleaving enzyme, a DNA-modifying enzyme (e.g., DNA methyltransferase, terminal transferase, T4 DNA ligase, DNA polymerase), an enzyme that modifies DNA-associated proteins, a restriction enzyme, an endonuclease, a homing endonuclease, a transposase, type-II CRISPR-Cas9 protein, dsDNA Fragmentase.

98. The device of claim 91, further comprising at least one instrument that facilitates DNA cleavage and/or DNA release from the channel, optionally wherein the at least one instrument is selected from a sonicator, a nebulizer, a light source (which emits e.g., visible light, UV light, X-rays, gamma rays, or ionizing radiation), or any combination of two or more thereof.

99. A method of selecting parameters for extracting fragments of genomic DNA having a desired size metric from a cell or cell nucleus, the method comprising:
processing a sample comprising at least one cell or cell nucleus through the device of any one of the preceding claims, at least once using parameters that comprise
a concentration for each of said one or more enzymes; a number of said one or more enzymes;
a buffer composition for said digestion buffer; a digestion time; and
a digestion temperature;
determining a fragment size metric for a collected sample; and, based on said fragment size metric,
if said fragment size metric corresponds to the desired size metric, selecting the used parameters;
if said fragment size metric is lesser than the desired size metric, reducing said concentration, reducing said number, reducing said digestion time, changing said digestion temperature to reduce enzyme efficiency, changing said buffer composition to reduce enzyme efficiency, or a combination thereof until said fragment size metric is not lesser than the desired size metric; and
if said fragment size metric is greater than the desired size metric, increasing said concentration, increasing said number, increasing said digestion time, changing said digestion temperature to increase enzyme efficiency, changing said buffer composition to increase enzyme efficiency, or a combination thereof until said fragment size metric is not greater than the desired size metric.

100. The method of claim 99, further comprising inactivating the one or more enzymes, optionally wherein said inactivation comprises heat inactivation.

101. A method of isolating a cell or a cell nucleus from a sample, the method comprising
flowing the sample through the device of claim 91; and
collecting the cell or cell nucleus entrapped in the device.

102. The method of claim 101, further comprising lysing the cell membrane or cell wall but leaving the nuclear membrane intact.

103. The method of claim 101, further comprising washing the entrapped cell or the cell nucleus.

104. The method of claim 101, wherein the sample is selected from blood, plasma, lymph, saliva, urine, in vitro cell culture, and tissue homogenates, optionally wherein the tissue homogenate is a plant tissue homogenate.

105. A method of isolating a genomic DNA of a large size (e.g., intact gDNA, whole chromosome, or a fragment of gDNA greater than 500 kb), the method comprising processing a sample comprising at least one cell or cell nucleus through the device of claim 91, optionally wherein the device comprises micropillars that are collapsible, meltable, mechanically retractable, dissolvable, mechanically peeled off, mechanically bendable, and/or electrically bendable.

106. A method of isolating fragments of genomic DNA having a selected size metric from at least one cell or cell nucleus, comprising:
(a) processing a sample comprising at least one cell or cell nucleus through the device of claim 91; and
(b) collecting the fragments of genomic DNA isolated via said processing.

107. The method of claim 106, wherein the fragments of gDNA are produced via digestion with a DNA-cleaving enzyme, sonication, nebulization, altering flow conditions, light method, mechanical method, or a combination of two or more thereof.

108. The method of claim 106, wherein the method comprises using the parameters selected via the method of claim 99.

109. The method of claim 106, further comprising
(a) washing the entrapped cell or the cell nucleus prior to the lysis;
(b) washing the genomic DNA prior to digestion; and/or
(c) inactivating the one or more enzymes (e.g., heat inactivation).

110. The method of claim 106, wherein the fragments of genomic DNA are in the range of about 1 kb to 500 kb, optionally wherein the fragments of genomic DNA are 200 kb, 30 kb, 25 kb, 14 kb, 10 kb, or 8 kb.
